(19) United States
(12) Patent Application Publication
(10) Pub. No.: US 2019/0167407 A1
(43) Pub. Date: Jun. 6, 2019

(54) DEVICES AND METHODS FOR LUNG VOLUME REDUCTION

(71) Applicant: SHIFAMED HOLDINGS, LLC, Campbell, CA (US)

(72) Inventors: Alan SCHAER, San Jose, CA (US); Tom SAUL, Moss Beach, CA (US); Amr SALAHIEH, Saratoga, CA (US); Lilip LAU, Los Altos, CA (US); Don TANAKA, Saratoga, CA (US); Sid GANDIONCO, Campbell, CA (US); Hung HA, Campbell, CA (US); Arindam DATTA, Campbell, CA (US)

(21) Appl. No.: 16/321,321

(22) PCT Filed: Aug. 4, 2017
(86) PCT No.: PCT/US17/45530

§ 371 Date: Jan. 28, 2019

Related U.S. Application Data
(60) Provisional application No. 62/371,171, filed on Aug. 4, 2016.
(60) Provisional application No. 62/376,874, filed on Aug. 18, 2016.
(60) Provisional application No. 62/384,189, filed on Sep. 6, 2016.

(51) Int. Cl. A61F 2/04; A61F 2/95

(52) U.S. Cl. CPC A61F 2/04; A61B 1/2676; A61F 2/95

(57) ABSTRACT

Methods of treating COPD, including bronchoscopically positioning a lung anchor in lung tissue, positioning an outer implant member in pleural space proximate the lung anchor, and coupling the lung anchor to the outer implant member and thereby compressing lung tissue.

### CROSS REFERENCE TO RELATED APPLICATIONS

This application claims priority to the following U.S. Provisional Applications, each of which is incorporated by reference herein in its entirety: Application No. 62/371,171, filed Aug. 4, 2016; Application No. 62/376,874, filed Aug. 18, 2016; and Application No. 62/384,189, filed Sep. 6, 2016.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Lung volume reduction (LVR) is an important procedure in the treatment of emphysema or chronic bronchitis, a form of Chronic Obstructive Pulmonary Disease (COPD). COPD is the third leading cause of death in the United States. Emphysema is a type of COPD involving damage to the air sacs (alveoli) in the lungs. As it worsens, emphysema turns the alveoli into large, irregular pockets with gaping holes in their inner walls. This reduces the surface area of the lungs and, in turn, the amount of oxygen that reaches the bloodstream during each breadth. The damaged lung tissue additionally loses its ability to hold its normal shape and becomes hyper-inflated, thereby consuming a larger volume than comparable healthy tissue. Emphysema also slowly destroys the elastic fibers that hold open the small airways leading to the air sacs. This allows these airways to collapse upon exhalation, trapping air in the lungs. Treatment may slow the progression of emphysema, but it can't reverse the damage.

Emphysema is often classified as to how uniformly diseased tissue or how uniformly the diseased state of the tissue is distributed through the lung. The two extremes are heterogeneous, where there are distinct pockets of diseased tissue separated by healthier tissue, and homogeneous, where the distribution of the diseased state of the tissue is more uniform. When there is a heterogeneous presentation, it is useful to reduce the volume of the most diseased area of a lung. When the presentation is homogeneous it is useful to treat a portion of the most diseased lobe of the lung.

There exists a need for minimally invasive treatments intended to bring relief to patients suffering from the stages of emphysema where diseased portions of the lung no longer efficiently contribute to the oxygenation of the blood, but instead provide a hindrance to lung function and capacity.

### SUMMARY OF THE DISCLOSURE

Some aspects of the disclosure herein relates to apparatuses and methods which provide for minimally invasive treatment via LVR in patients suffering from emphysema by providing mechanical compression of the emphysematous tissue. This compression serves to reduce the volume occupied by the emphysematous tissue. Additionally, the compression of diseased tissue restores some of the lost compliance or elasticity of the original tissue and allows for the distal airways to remain open during exhalation, thereby allowing the release of trapped gas from within the healthy tissue. These procedures provide the benefits of surgical lung volume reduction while minimizing the risks associated with the far more invasive surgical procedure.

Some of the apparatuses in this disclosure comprise an anchoring system which in turn comprises at least two anchors connected to one another by a tethering structure, wherein the systems can be configured such that the distance between the two anchors can be decreased. In some embodiments the two anchors are comprised of at least a proximal anchor, at least a distal anchor, the at least one distal anchor and the at least one proximal anchor connected to one another by a tether, and a mechanism to decrease the distance between the proximal and distal anchors. In some embodiments there are more than one distal anchors connected to a proximal anchor. In an alternate embodiment, the two anchors will be distal anchors and the proximal anchor will be the interface between the tether and a bifurcation in the bronchi. In many embodiments the distal anchor will be a fixation anchor designed to affix to the surrounding tissue, typically the wall of an airway, and in some cases additionally the tissues surrounding the airway.

In some embodiments the distance between two anchors may be adjusted by shortening the tether, in others by reducing the amount of tether between the two anchors. For the purposes of discussions herein foreshortening will describe either means of reducing the distance between anchors spanned by a tether. In some embodiments the distance between an at least one proximal and one or more distal anchor(s) is adjustable such that the distance may be increased, or decreased. Yet other anchor embodiments allow for the release of the tether completely. A number of embodiments described in which the tether is shortened follow. The proximal anchor comprises a way of twisting the tether on itself such that the tether winds on itself, thereby foreshortening. The tether can comprise a spring which on deployment shortens. Some embodiments in which the distance between two anchors is reduced by reducing the length of tether between two anchors are as follows. The proximal anchor comprises a mechanism of winding the tether onto a spool. The tether is pulled through a catch mechanism comprised in the anchor. Additionally the tether comprises a feature which interfaces with the catch mechanism. The tether is comprised of a material which can be caused to shrink, such as by denaturation resulting from heating or a pH change, after deployment. Twisting or spooling of the tether and thereby managing any and all excess tether length that may result from the tensioning and foreshortening of the tether on implementing a lung volume reduction reduces the likelihood of the anchoring system causing an inflammatory response within the lung. Once the volume of the lung is reduced in the desired area, the remaining portion of the lung continues to function. This dynamic motion could exacerbate any local damage or inflammatory response that excess tether or protruding features may cause.

As used herein a fixation anchor is a device that is designed to be affixed to an airway. Such anchors comprise a fixation mechanism that fixes the anchor to the airway wall. In some embodiments the fixation means is a mechanical aspect where fixation results from a mechanical interference with the airway wall. Mechanical embodiments may pierce the airway wall, rely on local expansion of the airway, rely on the branching characteristic of the airways, rely on the alveolar interface at the terminus of the airways. In alternate embodiments the fixation may be by adhesive mechanisms, and in others it use combinations of the above.

Some embodiments presented herein use a spike for fixation. The spike can be incorporated into the anchor such that, when deployed, tensions applied to the spike by the anchoring system, as a distal and proximal anchor are drawn together, will drive the spike into, and maintain the spike in, the airway wall. In such embodiments the spikes may be configured such that upon release form a delivery device the spikes will move from a delivery configuration, in which the spikes are directed at an angle roughly along the longitudinal axis of the anchor, to a delivered configuration in which the spikes are directed at least partially radially outward. In other embodiments the spikes may be maintained in the delivery configuration by a removable wire or tab which is removed at the time of deployment. Such embodiments comprise an actuable fixation mechanism. In some embodiments the spikes may be barbed such that once the tip passes through the airway wall the barb inhibits the ability of the airway wall to slip off the spike. In yet other embodiments the distal fixation mechanism may comprise the whole anchor. Such an embodiment is comprised in a tagging fastener where the end of the tether comprises the fixation anchor. In a tagging fastener the fixation anchor portion of the tether is “T” shaped. During deployment the top of the “T” is folded parallel to the stem of the “T” and is passed through the wall of an airway. After passing the end through the airway wall it relaxes into its deployed state where it takes the shape of the “T”. The top of the “T” now locking the tether to the airway. In some embodiments the tether may be terminated by a volume of porous material which is saturated by an adhesive delivered via a lumen in the tether.

In alternate embodiments the fixation mechanism is purely mechanical in nature, where the airway wall is not breached by the fixation means. Such embodiments comprise any of the following. Expanding structures such as spiral springs which expand the airway wall to a point where the structure is unable to slip. An anchor comprised of an array of interconnected distal airways filled with an adhesive or expanding material such as a PMMA or a collagen plug.

In some embodiments each proximal anchor will connect to one distal anchor. In others, each proximal anchor will connect with one distal anchor. In yet other embodiments the anchoring features will be distributed along the entire extent of the anchoring structure.

In some embodiments the proximal anchors will be placed in tissue less diseased than that in which the distal anchors are placed. Such an embodiment will be particularly useful in treating a more heterogeneous presentation of the disease. In other embodiments the distal anchors will be placed in tissues at the borders of diseased tissue also useful in treating a more heterogeneous presentation. In other embodiments the anchors will be placed in airways surrounded by tissues of a relatively uniform disease state such as in a homogeneous presentation where the tissues of a particular lobe are of a relatively uniform diseased state, but the particular lobe is more diseased the other lobes of the lung.

In some embodiments of this disclosure the anchors will be drawn together in a sequential fashion. Such a sequential foreshortening minimizes stress gradients across the volume reduced tissue both during the procedure and after completion of the procedure thereby reducing the risk of tears arising in the tissue and resultant loss in the total volume reduction. In a sequential procedure multiple anchor systems and or anchors within an anchor system will be foreshortened in an incremental fashion. Each tether will be foreshortened incrementally by an amount less than the total expected for the end LVR. In this way each tether will be foreshortened multiple times during the procedure. Alternatively, sequential may mean foreshortening one tether at a time.

In some instances such as when treating heterogeneous emphysematous tissue where some anchors can be placed in the peripheral healthier tissue at the borders of the more diseased tissue, and others are placed within more diseased tissues, the sequential procedure will allow the peripheral anchors to be drawn up first followed by those in the less healthy tissue. In such a situation it can be desirable to draw in the boundary tissues more than the central anchors to start. As the healthier tissue compresses in on the less healthy tissue less force will be required to draw in the less healthy tissue thereby reducing the risks of tears in the tissue. In situations where the tissue is of more uniform quality, adjusting each anchor by a consistent amount and cycling through all of the anchors multiple times will be more advantageous.

In any procedure if tears are observed either by imaging or other means to be described, the foreshortening of individual anchors can be reversed relieving the stress gradients across the tissue. In such situations additional anchors may also be placed. Such a procedure is facilitated when performed under Fluoro or other medical imaging system.

Prior to any procedure a pre-evaluation can be performed to facilitate the eventual procedure. Such a pre evaluation can comprise any of the following procedures. Imaging procedures such as CT, standard Xray, Fluoroscopy (Fluoro), MRI, or ultrasound. Functional evaluations such as FEV1, RV, FVC, TLC, or other lung function test. Additionally tests which provide insights into the compliance, both dynamic and static, and or density distribution of the lung tissue will be useful. For the purpose of characterizing density and compliance an intrabronchial ultrasound will be useful.

After the pre-procedure evaluations are concluded a planning step can be performed. Such a step may be performed at the time of the LVR procedure and incorporate additional evaluations or it may be performed prior to the LVR procedure. The planning step will comprise some combination of the following. The identification of regions to be treated based on, density and or compliance as determined by medical imaging. An intrabronchial ultrasound can be particularly useful in such determinations, especially when preformed during the procedure. The identification of boundary between emphysematous and normal tissue using any of the techniques described herein. A determination of the number of and location of devices to be placed within and around or at the boundary of the emphysematous tissue. A determination of an initial goal for amount of tissue reduction predicated on any of the evaluations described herein.

A stepwise reduction may be performed in addition to or in combination with sequential reduction. In a stepwise reduction a period of time is allowed to pass prior to each incremental reduction, where each incremental reduction may comprise a foreshortening of all tethers or some subset of all of the tethers. A stepwise reduction may comprise any combination of the following. A stepwise reduction predicated on a healing response. Such a procedure would incorporate some combination of the following steps. Implant a set of anchors then apply coordinated sequential loading, load or displacement, to each anchor. The target magnitude of the loading or displacement increments characterized by any of the evaluations performed previously or elsewhere herein. The amount of displacement or loading applied determined using flouro, force measurements or torque measurements. Allow for tissue stabilization for a period of 5 minutes to 3 months (or more such as out to one or more years) depending on the magnitude of the healing response desired. Repeat the process until the desired LVR is achieved.

Alternatively or in combination the stepwise procedure may be predicated on allowing for an initial ingrowth/fixation of the anchors. Such a procedure would comprise some combination of the following steps. Implant anchors and allow tissue ingrowth to stabilize for a period of 7 days to 3 months. Then apply coordinated sequential loading load or displacement to each anchor. The target magnitude of the loading or displacement increments characterized by any of the evaluations performed previously. The amount of displacement or loading applied determined using flouro, force measurements or torque measurements. Allow for tissue stabilization for a period of 5 minutes to 3 months (or more such as out to one or more years) depending on the magnitude of the healing response desired. Repeat the process until the desired LVR is achieved. The process can be repeated until the desired outcome is achieved. In some circumstances adjustments may be repeated at time periods of one year or more to accommodate further deterioration of the emphysematous condition.

In some embodiments the device is implanted but lung volume is not immediately reduced. This can be done to allow initial ingrowth/fixation as discussed herein with risk of tearing of tissue. Methods of reducing lung volume can therefor include endobronchially delivering an anchoring device to a location within the lung within a delivery device, the anchoring device comprising a distal anchor, a proximal anchor, and a tether extending between the distal and proximal anchors, the device configured such that the distance between the distal and proximal anchors measured along the tether can be increased or decreased and then maintained after release of the anchoring device from a delivery device, deploying the anchoring device completely out of the delivery device, and removing the delivery device from the lung without increasing or decreasing the distance between the proximal and distal anchors. After a period of time that has sufficiently allowed fixation or ingrowth, the lung volume is then reduced.

In stepwise and sequential procedures the number reductions can be predicated on the pre evaluation and or pre procedure data. Procedure planning and pre-characterization of tissue quality can improve procedure outcome and is an important part of such procedures.

Some of the procedures described herein are facilitated by apparatus comprising some combination of the following. A flexible multi-lumen catheter suitable for use in an airway. Catheters comprising balloons or multiple balloons which may be used as temporary or permanent anchoring devices. Balloons which are permeable and allow for an adhesive to permeate through the balloon wall. Medical grade tissue adhesives or bioadhesives for use in fixing anchoring components. Space filling bio-materials such as gels and solids such as epoxies. Catheters comprising a means for penetrating the airway wall such as a directable hypo-tube capable of piercing the wall of an airway and delivering a mechanical anchor to a target area, and or delivering an adhesive or space filling material to a target area. Catheters comprising optical means such as a flexible fiber-optic fiber or LED capable of light by which the adhesive may be cured and other means for curing adhesives and space filling materials. In some embodiments a flexible fiber-optic tube capable of delivering both a light-curable adhesive and the light by which the adhesive may be cured may be used. A flexible catheter and balloon system capable of delivering an adhesive and providing a specified vacuum force to a target area. Such systems capable of releasing the implant portions of any anchoring system.

Some of the apparatus may additionally comprise devices capable of performing diagnostics such as the following. An intra-bronchial ultrasound transducer for use in characterizing density or compliance of local tissue. Alternatively, electrodes may be provided to allow for electrical impedance (EI) measurements as a way of characterizing tissue electrical impedance as a function of hyper inflated state and or changes in tissue electrical impedance as a function of tissue compression arising from the lung volume reduction. In other embodiments electrical impedance changes between multiple anchors may be used to indicate appropriate compression or tearing of tissues between the multiple anchors. In these embodiments the methods can include endobronchially positioning a tissue characterizing device within the lung, activating the characterizing device at one or more locations in the lung, and endobronchially deploying a distal anchor of a lung volume reduction device within the lung at a target location after determining that the target location of the lung is emphysematous tissue.

To enhance the efficacy and safety of the sequential and stepwise foreshortening procedures anchors may have load monitoring means incorporated into their structure. Alternatively load may be derived from the amount of spiraled tether as noted by fluoroscopy. Alternatively the amount of torque required to foreshorten a tether will indicate the forces acting on the tether. In such systems the force to displacement behavior may be monitored to indicate how the tissue under volume reduction is responding. When tissue begins to tear as noted by a decrease in load associated with a foreshortening the user may back off and lengthen that tether thereby removing tension. Alternate surrounding tethers or new tethers can be placed in the surrounding tissues. Alternatively or in combination some form of stepwise procedure may be instituted. In some embodiments the force displacement curves are displayed real time to the user. In some embodiments the expected maximum compression of portions of the lung to be treated will be predicted by density and or compliance measurements and these predictions used to inform the size of load or displacement increments to be applied during a sequential tether foreshortening procedure.

In some circumstances, such as when the treatment in a non-responder provides no or minimal clinically positive outcomes, it may be desired by the physician to return the patient to the pre-operative state, or as close as possible to it. Some embodiments include reducing the tension applied to the lung tissue. In other embodiments, the proximal anchor or the entering anchoring device can be removed.

One aspect of the disclosure is a device for reducing the volume of a lung, comprising: a distal anchor, a proximal anchor, and a tether extending between the distal and proximal anchors, the device configured so that the distance between the anchors measured along the tether can be increased or decreased and maintained after release of a delivery device.

In some embodiments of this aspect the device is further configured so that the distance between the anchors can be further increased or decreased after the device has been released from a delivery device.

In some embodiments of this aspect the device further comprises a tensioning controller that interfaces with the tether, the tensioning controller configured to be actuated to increase or decrease the distance between the proximal and distal anchors.

In some embodiments of this aspect a tether actual length between the anchors stays the same. The tether can be adapted to be reconfigured such that the distance measured along the tether between the anchors can be reduced. In some embodiments only a portion of the tether is configured to be reconfigured.

In some embodiments of this aspect the tether is configured to wind up on itself to decrease the distance between the anchors.

In some embodiments of this aspect the distal anchor is disposed at a distal end of the device, the proximal anchor disposed at a proximal end of the device, and the device does not include any other anchors disposed between the distal and proximal anchors.

In some embodiments of this aspect the distal and proximal anchors are expandable.

In some embodiments of this aspect at least one of the distal and proximal anchors has an electrode thereon.

In some embodiments of this aspect the device is configured so that as the distance between anchors changes, a tether axis remains in the same direction. The axis can remain in the same direction even though the tether changes configuration.

In some embodiments of this aspect the device is configured so that as the distance between anchors changes, the rotational orientation, out of a plane comprising the tether axis, of the distal anchor stays the same relative to the proximal anchor.

In some embodiments of this aspect the proximal anchor is configured to be collapsed and removed from the lung after it has been expanded towards an expanded configuration. The distal anchor can be configured to be collapsed and removed from the lung after it has been expanded towards an expanded configuration.

One aspect of the disclosure is a method of reducing the volume of a lung, comprising endobronchially deploying an anchoring device within the lung, the anchoring device comprising a distal anchor, a proximal anchor, and a tether extending between the distal and proximal anchors, the device configured such that the distance between the distal and proximal anchors measured along the tether can be increased or decreased and then maintained after release of the anchoring device from a delivery device; reducing the volume of the lung by decreasing the distance between the distal and proximal anchors; and maintaining the decreased distance.

In some embodiments of this aspect the method further comprises, after the positioning step, releasing the anchoring device from a delivery device and removing the delivery device from the lung without decreasing the distance between the proximal and distal anchors, wherein the reducing and maintaining steps are performed after the releasing and removing steps. The reducing and maintaining steps can be performed after a second delivery device is endobronchially positioned within the lung.

In some embodiments of this aspect, after the maintaining step, waiting a period of time during which the distance between the anchors is not changed, and after the waiting step, at least one of increasing or decreasing the distance between the proximal and distal anchors. The waiting step can comprise monitoring a characteristic of the lung. The waiting step can comprise waiting a period of time for at least one of the following to occur: tissue relaxation, tissue ingrowth into one or both anchors; and a healing response in the volume reduced tissue. The method can comprise, after the waiting step, decreasing the distance between the proximal and distal anchors to further reduce the volume of the lung. The waiting step can comprise waiting at least 2 minutes during which the distance between the anchors is not changed.

In some embodiments of this aspect decreasing the distance comprises increasing the tension in the tether.

In some embodiments of this aspect, after the maintaining step, increasing the tension in a second tether extending from a second distal anchor also positioned in the lung. Increasing the tension in a second tether can comprise increasing the tension in a second tether that is coupled to a second proximal anchor different than the proximal anchor. Increasing the tension in a second tether can comprise increasing the tension in a second tether that is coupled to the proximal anchor.

In some embodiments of this aspect the method further comprises endobronchially positioning a second anchoring device within the lung, the second anchoring device comprising a second distal anchor, a second proximal anchor, and a second tether extending between the second distal and second proximal anchors, the second device configured such that the distance between the second distal and second proximal anchors can be increased or decreased and then maintained after release of the second anchoring device from a delivery device.

In some embodiments of this aspect decreasing the distance comprises causing at least a portion of the tether to wind up on itself.

In some embodiments of this aspect the method further comprises, prior to the deploying step, characterizing a physical quality of lung tissue using an endobronchially placed characterization device. Characterizing a physical quality of a portion of the lung can comprise characterizing a physical quality of the lung that is indicative of emphysematous tissue. The physical quality can be at least one of tissue compliance and tissue density. After the characterizing step characterizes the portion of the lung as emphysematous tissue, the method can include anchoring the distal anchor in the emphysematous tissue. The characterizing step can comprise measuring the electrical impedance of the lung tissue. The method can also include determining a maximum tension to apply to the distal anchor using the results of the characterizing step.

In some embodiments of this aspect decreasing the distance between the distal and proximal anchors comprises actuating a tension controller secured to the proximal anchor.

In some embodiments of this aspect the method further comprises, after the reducing step, increasing the lung volume by adjusting the anchoring device. Adjusting the anchoring device can comprise increasing the distance between the anchors. Adjusting the anchoring device can comprise removing the proximal anchor from the lung. Adjusting the anchoring device can comprise removing the distal anchor from the lung.

One aspect of the disclosure is a method of reducing lung volume, comprising endobronchially positioning a tissue characterizing device within the lung; activating the characterizing device at one or more locations in the lung; and endobronchially deploying a distal anchor of a lung volume reduction device within the lung at a target location after determining that the target location of the lung is emphysematous tissue. The activating step comprises activating an electrical impedance device, wherein the distal anchor includes an electrode thereon. The activating step can comprise activating an electrical impedance device, wherein a delivery device includes an electrode thereon. The activating step can comprise activating an ultrasound device on a delivery tool.

One aspect of the disclosure is a method of reducing lung volume, comprising endobronchially reducing a volume of lung with a lung volume reduction device; waiting a period of time at least 2 minutes without further reducing the volume of the lung; and after the waiting step, further reducing the volume of the lung.

One aspect of the disclosure is a method of reducing lung volume, comprising endobronchially reducing a volume of lung with a lung volume reduction device; after the reducing step, waiting a period of time without further reducing lung volume sufficient to allow at least one of tissue relaxation, tissue ingrowth into a part of the device; and a healing response in the volume of reduced tissue to occur; and after the waiting step, further reducing the volume of the lung.

One aspect of the disclosure is a method of reducing the volume of a lung, comprising endobronchially delivering an anchoring device to a location within the lung within a delivery device, the anchoring device comprising a distal anchor, a proximal anchor, and a tether extending between the distal and proximal anchors, the device configured such that the distance between the distal and proximal anchors measured along the tether can be increased or decreased and then maintained after release of the anchoring device from a delivery device; deploying the anchoring device completely out of the delivery device; and removing the delivery device from the lung without increasing or decreasing the distance between the proximal and distal anchors.

As set forth above, a need exists to reduce the volume of hyperinflated diseased lung tissue such that inspired air reaches more healthy lung tissue. In addition, reducing compression of the lung from the hyperinflated regions on the diaphragm and chest wall helps to improve lung mechanics. Additionally, retensioning the lung tissue helps reduce trapped air by preventing airway collapse during exhalation. Hyperinflated regions may enlarge spaces within the lung parenchyma, but also may include blebs or bulla on the outer surface of the lung. In many cases compression of blebs or bulla may not be possible with mechanical retensioning from within the lung only. It may be desired or necessary to reduce the volume of these blebs and bulla making use of a compressive force from the outer visceral surface of the lung toward one or more securing points within the lung. Importantly, this compression should occur without dissecting or otherwise breaching the thin membranous outer tissue of the lung which can lead to pneumothorax.

A further aspect of this disclosure includes methods for coupling an implant that is placed into the pleural space on the outer surface of the lung to one or more anchors implanted within the lung, which can optionally be placed bronchoscopically. Coupling of the outer implant to the inner implanted anchor(s) allows the ability to compress lung volume, particularly that comprising blebs and bulla, between the outer implant and inner implant. The coupling also improves the ability of the inner implant to be secured within the lung tissue, which is often diseased and difficult to support significant tension from the inner anchor alone. Using a tension tether line attached to the inner implant, the coupled outer implant and inner implant may thus also be tensioned as a unit to compress tissue between this coupled unit and a more proximal bronchoscopically-implanted anchor. The methods of placement and use and exemplary system and device embodiments are provided below.

One aspect of the disclosure is a lung volume reduction device, comprising: a distal anchor, a proximal anchor, and a connector extending between the distal and proximal anchors, the distal anchor comprising a body with a longitudinal axis and an extension member with a first end secured to the body, wherein in a deployed configuration the extension member extends from the body and away from the longitudinal axis.

In a delivery configuration, the extension member can extends generally parallel to the longitudinal axis.

The connector can be secured to the distal anchor at a location that is 10%-50% of a length of the distal anchor, and optionally less than 50% of the length (not extending from a midpoint of the length).

The distal anchor can be formed from a tubular element. The distal anchor can also include one or more piercing elements adapted to pierce lung tissue.

One aspect of the disclosure is a lung volume reduction device, comprising: a distal anchor, a proximal anchor, a connector extending between the distal and proximal anchors, and a distal anchor extension extending from a body of the distal anchor at a location that is not the mid-point of a length of the body (optionally at a location that is 10-50% of the length of the anchor). The distal anchor extension can be part of the distal anchor. The distal anchor extension can be part of the connector. The distal anchor can be formed from a tubular element. The distal anchor can also include one or more piercing elements adapted to pierce lung tissue.

One aspect of the disclosure is a lung volume reduction device, comprising: a distal anchor, a proximal anchor, and a connector (optionally a tether line) extending between the distal and proximal anchors. The tether line can extend from a location along the length of the distal anchor and extend proximally through a ratchet feature on the proximal anchor. The tether line can be releasably secured to the proximal anchor using a ratchet system that is adapted to automatically hold the tether line to the proximal anchor in a secure relationship when tension proximal to the proximal anchor is released, but allows the tether line to move proximally when tensioned from a proximal location. The ratchet can be formed from a tubular element having an upper surface and lower surface defined by opposite sides in the radial direction, the upper surface having a material removed along three sides to form a pawl having a tip, the tip oriented in the proximal direction, the lower surface having material removed to form a window larger than the pawl, the pawl being bent to pass through the window and beyond the diameter of the lower surface. The tubular element can be formed of nitinol having a wall thickness of at least 0.005″. The bend in the pawl can be formed by heat setting the nitinol. The connector can pass through the center of the tubular element and around the tip of the pawl. Tensioning the connector proximally can at least partially straighten the pawl to allow the connector to move more freely. Tensioning the tether line distally can at least further bend the pawl through the lower window to further constrain the movement of the connector.

### BRIEF DESCRIPTION OF THE DRAWINGS

FIGS. 1A-1C illustrates an exemplary treatment device comprised of three components.

FIG. 2 shows the airway anchor in a cutaway view.

FIG. 3 identifies structures of the lung for the purposes of simplification. Additionally, a portion of the parenchyma is afflicted with emphysema.

FIG. 4 shows a bronchoscope tracked into the airway leading to the emphysematous tissue to be treated.

FIG. 5 a distal anchor is deployed from the treatment device.

FIG. 6 the delivery sheath is withdrawn further back into the bronchoscope to deploy the proximal anchor.

FIG. 7 a drive shaft engages with the interface of a socket in the proximal anchor.

FIGS. 8A and 8B illustrate drive shaft rotation transmitted through the socket and into the tether, with the distal anchor drawn into closer proximity to the proximal anchor.

FIG. 9 a volumetric reduction in the emphysematous portion of the lung can be observed.

FIGS. 10-16 illustrate an exemplary mechanism configured to hold and adjust tension on a tether.

FIGS. 17-19 illustrates an embodiment for a method of reducing the volume of a lung by positioning a plurality of separated treatment devices within the lung.

FIGS. 20-25 illustrate methods of use that can be used when placing a plurality of distal anchors in different lumens for lung volume reduction.

FIGS. 26-28 illustrate various spring-like tether embodiments

FIGS. 29-32 describe alternative methods and devices for lung volume reduction.

FIGS. 34A-42E illustrate additional non-traumatic anchors for lung volume systems, devices, and methods of use.

FIGS. 43-44 represents an exemplary embodiment wherein the distal anchor is formed from a tubular element and includes an extension.

FIGS. 45-48 illustrate tubular variations on anchor and tether embodiments.

FIG. 49 shows the proximal anchor formed from a tubular element.

FIG. 50 presents the distal anchor, tether line, and proximal anchor as laser cut or etched from a single metal tube.

FIGS. 51A-51C show how the tether line may be ratcheted through an alternative proximal anchor.

FIGS. 52A-52C illustrate a mechanism for how the pawl may be disengaged from the line.

FIG. 53 illustrates a pulmonary anatomy comprising lung bronchus (or airway) and surrounding lung tissue within chest wall.

FIG. 54 depicts a pleural delivery catheter (PDC) containing outer implant introduced into the pleural space.

FIGS. 55-62 illustrate how more than one distal anchor may be deployed within the bronchi.

FIG. 63 illustrates an embodiment of the coupled outer implant and distal anchor.

FIG. 64 uses MCE that have north/south axes approximately perpendicular to the coupled tissue.

FIG. 65 illustrates how the outer implant may be a linear array of MCE constructed upon a molded matrix or netlike structure.

FIGS. 66 and 67 illustrate how the outer implant may be constructed with multiple MCE in a 2-D array on a net-like structure or fenestrated elastic film.

FIGS. 68A-70B describe each MCE as a sphere of approximately 2 mm in diameter having a north and south magnetic axis (similar to the earth).

FIGS. 71 and 72 provide an embodiment where an atraumatic catheter may be advanced into a distal diseased lung tissue region.

### DETAILED DESCRIPTION OF THE DISCLOSURE

The disclosure describes methods, devices, and systems for reducing the volume of a lung.

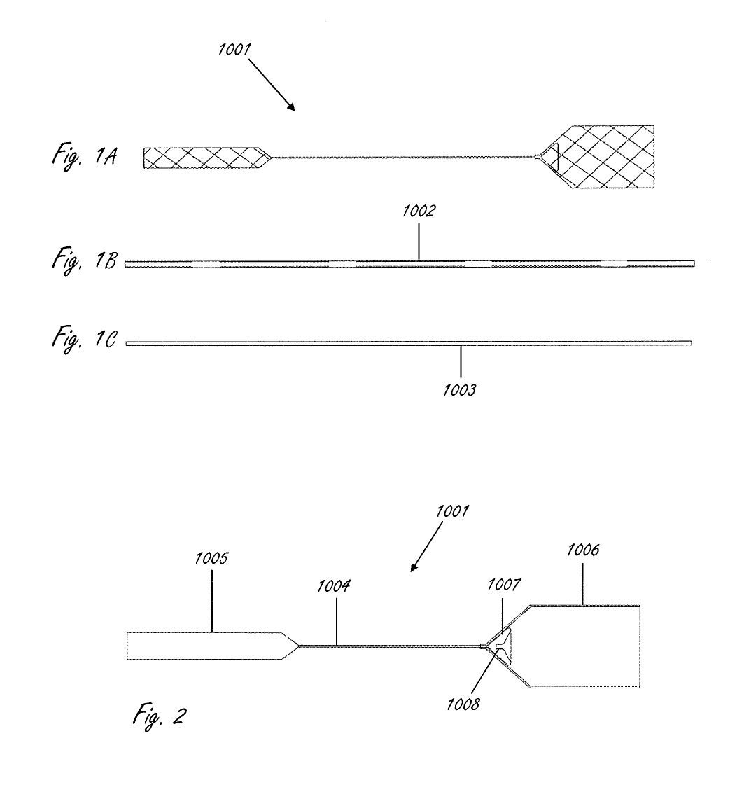

FIGS. 1A-1C and 2 illustrate an exemplary embodiment of a lung volume reduction apparatus. The embodiment in FIGS. 1A-1C and 2 is an example of a device for reducing the volume of a lung that includes a distal anchor, a proximal anchor, and a tether extending between the distal and proximal anchors, the device configured so that the distance between the anchors measured along the tether can be increased or decreased and maintained after release of a delivery device. Apparatuses and devices configured and/or adapted to reduce the volume of a lung may also be referred to herein as “treatment devices.” The apparatus shown in FIGS. 1A-1C includes three components. The first component is an airway anchor (1001) as shown in FIG. 1A. An “airway anchor” may also be referred to herein as an “airway anchoring device” or other derivative. The airway anchor is designed to be collapsed into a small profile and delivered by the second component, a delivery sheath (1002), which is illustrated in FIG. 1B. The third component of the apparatus is a drive shaft (1003), shown in FIG. 1C, configured to tighten the airway anchor (1001) once the airway anchor is positioned in its target location. The act of “tightening” as used herein may also be referred to herein as “tensioning.” Delivery sheath (1002) shown in FIG. 1B includes a lumen configured to house therein a plurality of separate anchoring devices, the plurality of anchoring devices positioned along the length of the lumen. That is, the anchoring devices are disposed within the lumen axially from one another rather than radially. In this embodiment an inner lumen of delivery sheath includes four anchor housing regions, each for receiving an anchor therein. The distal two regions thus receive the distal and proximal anchors of a first anchoring device, and the proximal two regions receive the distal and proximal anchors of a second anchoring device. The lumen can be configured to stably house any number of anchoring devices therein. The use of multiple anchoring devices is described below.

FIG. 2 illustrates a sectional view of airway anchor (1001) from FIG. 1A. The airway anchor (1001) includes an actuatable distal anchor (1005), which is configured to be expanded from a first compressed configuration that allows it to be collapsed within delivery sheath (1002) for delivery to an expanded configuration for engaging an airway wall. Such exemplary expansible structures may include laser cut nitinol, braided nitinol, inflatable structures, and the like. The distal anchor (1005) may comprise a plurality of tines (as described further below) to maintain traction with the airway wall. The airway anchor (1001) also includes tether (1004) that is fixedly attached to the distal anchor (1005) on one end, and attached to, while maintaining rotation freedom from, the proximal anchor (1006). In this embodiment tether (1004) is constructed of material that maintains a high tensile and torsional strength to prevent breakage. In this embodiment tether (1004) is also somewhat flexible, so that upon twisting it is capable of winding itself into a non-straight configuration, and therefore becoming shorter without breaking or transmitting excessive torque to the distal anchor.

In some embodiments the tether is any of or a combination of Dacron®, Dyneema®, Spectra and Kevlar®. The tether can be a wide variety of common fishing line. In some embodiments braided Dacron® can be used. The tether can be a monofilament, a nanofilament (i.e., hundreds of longitudinal strands), as well as braided.

Adequate volume reduction may be achieved with reductions in proximal to distal anchor distance associated with less than a few percent of initial length, especially when initial tether length is great, and up to 100%, especially when initial tether lengths are short. Large reductions may be effected in multiple smaller increments with time periods allowed between reductions for tissue relaxation or healing as is described elsewhere herein. The effect of the sum of local anchor adjustments will typically support lobal lung volume reductions of up to 30%, more typically 20%, and some situations, such as but not limited to when tissue is particularly friable, less than 20% perhaps only a few percent. Local tissue volume reductions may be even greater.

In some embodiments the tether winds up on itself when twisted. In these embodiments the tether may wind up in a very controlled and repeatable configuration, or it may wind up and take on a variety of configurations. In either case the winding is reliable and repeatable, even if the wound-up configuration is not completely predictable. In some embodiments the tether could be material used for fishing line, that when twisted will wind up, or bunch up, on itself.

The airway anchor (1001) also includes proximal anchor (1006). Similarly to the distal anchor (1005), proximal anchor (1006) is configured to be expansible from first compressed configuration so that it can fit within the delivery sheath (1002), to a larger expanded configuration for engaging the airway wall. Such expansible structures may include laser cut nitinol, braided nitinol, or inflatable structures and the like. The proximal anchor (1006) may optionally include a plurality of tines (as described further below) to maintain traction with the airway wall. The anchoring device also includes socket (1007), which is secured to the proximal anchor (1006), and which is mechanically connected to tether (1004), but allows the tether to rotate within and with respect to the proximal anchor. The socket (1007) includes an interface (1008) configured to receive drive shaft (1003) therein. The drive shaft and interface are configured such that the drive shaft, when positioned in the socket, is rotationally fixed with respect to the socket. Rotation of the drive shaft thus causes rotation of the socket. This arrangement allows the user to engage the drive shaft (1003) into the socket (1007) of the proximal anchor (1006), and twist the tether by twisting the drive shaft. The act of twisting the tether changes the configuration of the tether from a straight configuration to a non-straight configuration, resulting in the distal and proximal anchors being drawn together and the distance between the anchors measured along the tether reduced.

FIGS. 1A-1C and 2 illustrate a merely exemplary lung volume reduction device and additional exemplary devices are descried below. FIGS. 3-9 illustrate an exemplary method of using the device shown in FIGS. 1A-1C and 2.

FIG. 3 illustrates a portion of a lung, a complex organ composed of airways, blood vessels, alveolar tissue, lymphatic tissue among other structures. In this section, only major airways (1009) and parenchyma (1010) will be referred to for the purposes of simplification. Major airways (1009) refers to the bronchi that carry air to and from the parenchyma (1010) for oxygen transport. The parenchyma (1010) refers to all other structures in the lung, a majority volume of which is alveolar tissue. In FIG. 3, both major airways (1009) and parenchyma (1010) are present. Additionally, a portion of the parenchyma (1011) shown with shading is afflicted with emphysema.

FIG. 4 illustrates an initial step in the delivery of a treatment device to a target location within the lung. Bronchoscope (1012) has been navigated and tracked into the airway leading to the emphysematous tissue to be treated. Once in place, delivery sheath (1002) is tracked distally into the emphysematous tissue. The delivery sheath should be advanced as far as practical, while avoiding potentially rupturing the parenchyma.

In some embodiments the distal end of the delivery sheath will comprise a tissue evaluation device which is used to identify emphysematous tissue. One such evaluation comprises the measurement of the electrical impedance of the tissue. Alternative means include but are not limited to, ultrasonic, and optical means. Electrode elements 1131 comprised on the distal end of the delivery sheath (1013) are used to query the adjacent tissue as the device is delivered down the bronchi. If emphysematous tissue is observed, as would be the case in the illustration of FIG. 4, a distal anchor may be placed.

FIG. 5 illustrates a subsequent step (not necessarily immediately after) in the delivery of the device. As shown in FIG. 5, distal anchor (1005) has been deployed from the delivery sheath and has expanded into or towards its expanded configuration. Methods of deploying an expandable anchor from a delivery sheath are known, such as retracting a delivery sheath relative to an anchor whose position in maintained. The distal anchor optionally has a plurality of tines (i.e., sharp protrusions that puncture, hook into, or otherwise obtain traction) that engage the airway wall in which the anchor is deployed. In some embodiments there can be 4-300 barbs or tines that engage the vessel wall, with larger numbers being preferred (but not required) because the load carried by the anchor will be better distributed as more tines are involved.

Distal anchor (1005) is configured to radially expand in response to expansion of the airway in which it is anchored. The anchor should be capable of 100%-700% of the maximum expansion expected of the airway in which it is deployed. Providing such expansibility will prevent the airway from expanding to a diameter that exceeds the ability of the anchor to remain engaged with the airway, resulting in a loss of anchoring.

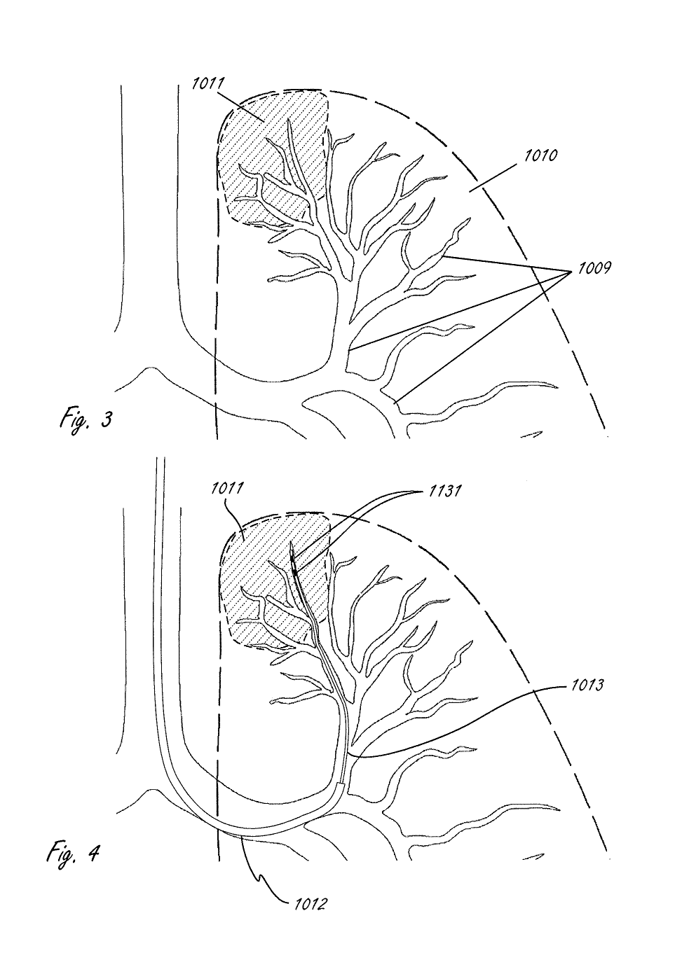

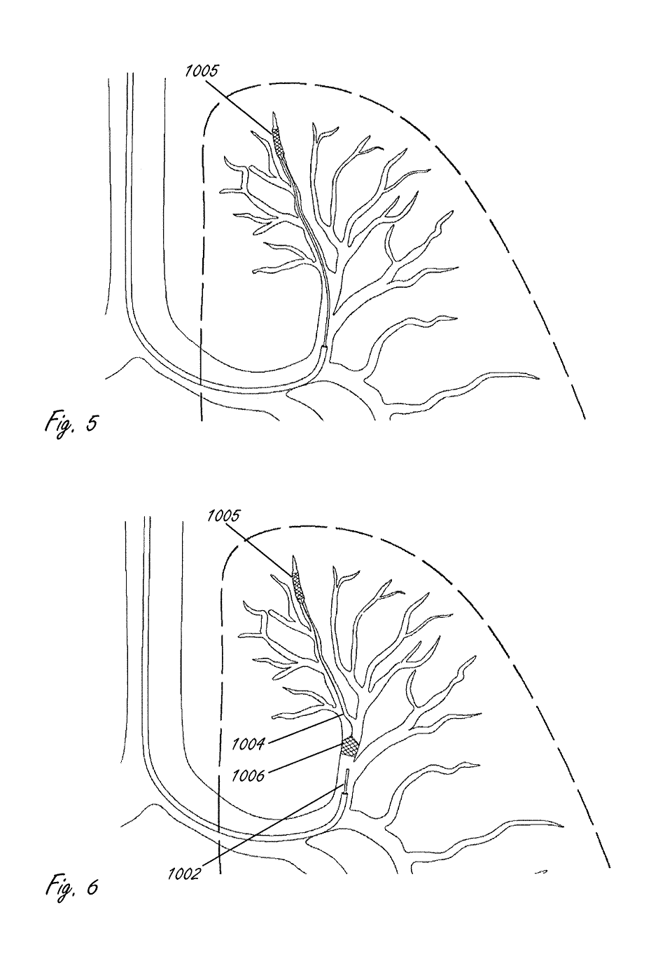

A subsequent step (but not necessarily immediately after), as shown in FIG. 6, is to deploy the proximal anchor (1006) from the delivery sheath and expanding proximal anchor (1006). Tether (1004) can be seen extending between the distal anchor (1005) and the proximal anchor (1006). The delivery sheath (1002) can be withdrawn proximally to deploy the proximal anchor (1006). The tether (1004) maintains a mechanical connection between the distal anchor (1005) and the proximal anchor (1006).

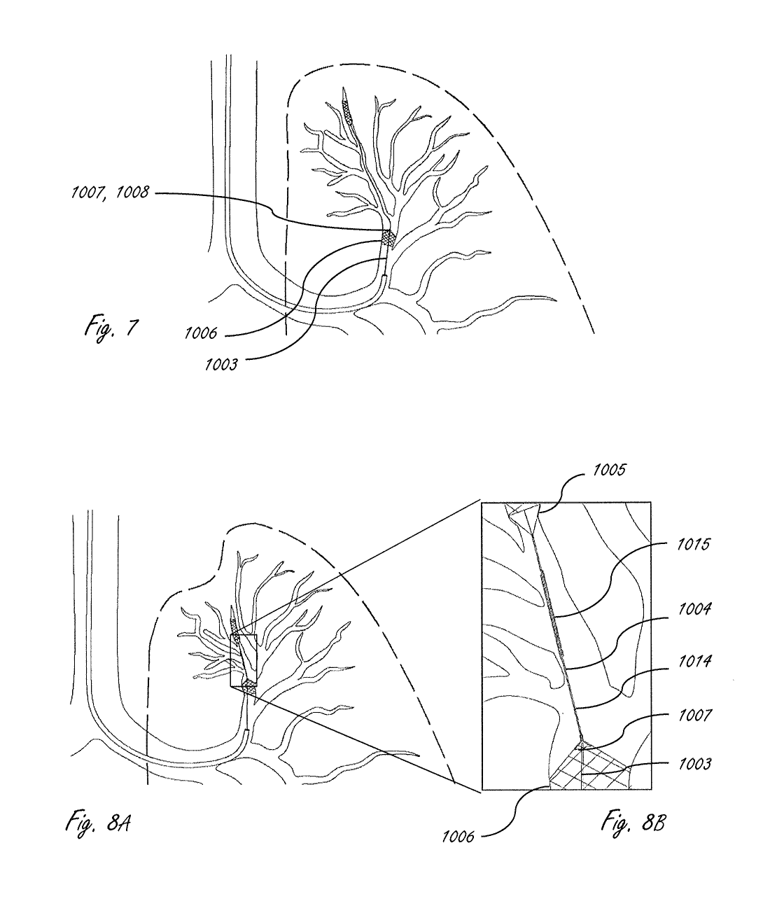

FIG. 7 shows, after the proximal anchor has been deployed at a target location, a drive shaft (1003) can then be tracked through the bronchoscope or through the sheath contained within the bronchoscope so that its distal end engages with the interface (1008) of the socket (1007) in the proximal anchor (1006). In this embodiment, when the drive shaft engages with interface (1008), the drive shaft and the socket are rotationally coupled.

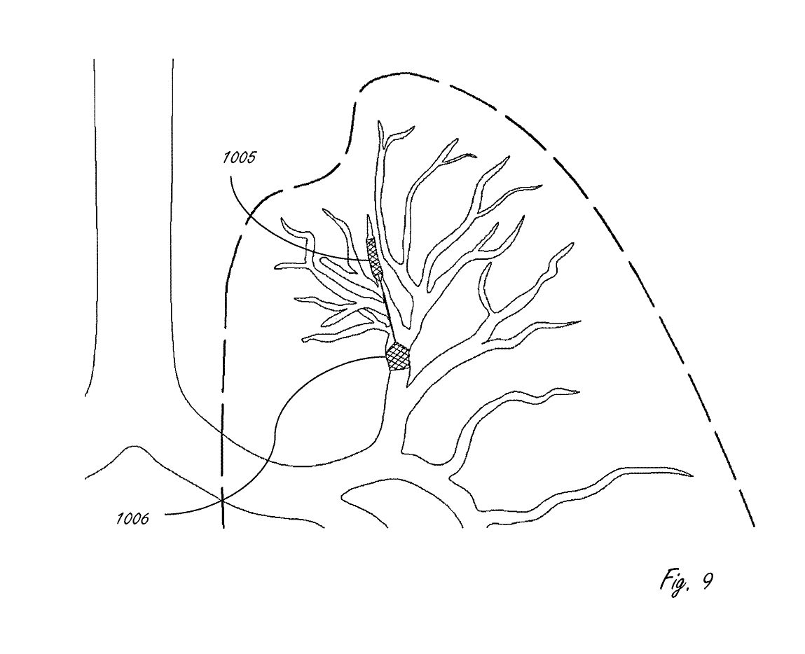

As shown in FIGS. 8A and 8B, the user then actuates (in this embodiment by rotating) the drive shaft (1003), causing the rotation to be transmitted through the socket (1007) and into the tether (1004). Rotating the drive shaft causes the tether to change configurations from a first configuration to a second configuration, which shortens the distance between the anchors. In this embodiment, as shown in the detailed view in FIG. 8B, the actuation causes a first portion (1015) of the tether to coil up into a non-straight configuration. This act of assuming a non-straight configuration causes the distal anchor (1005) to be drawn towards the proximal anchor into closer proximity to the proximal anchor (1006). The shortening of the distance between the distal anchor (1005) and the proximal anchor (1006) measured along the tether collapses the tissue between the anchor and has caused a volumetric reduction in the emphysematous portion of the lung.

FIG. 9 illustrates the treatment device in place within the lung after the bronchoscope has been removed. At this stage the final outcome of the lung volume reduction procedure can be observed.

All of the additional methods, devices, and systems described in WO/2016/115193 are fully incorporated herein. Any of the methods, systems, and devices expressly described herein can integrate any aspect of any suitable method, device, or system from WO/2016/115193, as if those alternative embodiments were expressly included herein.

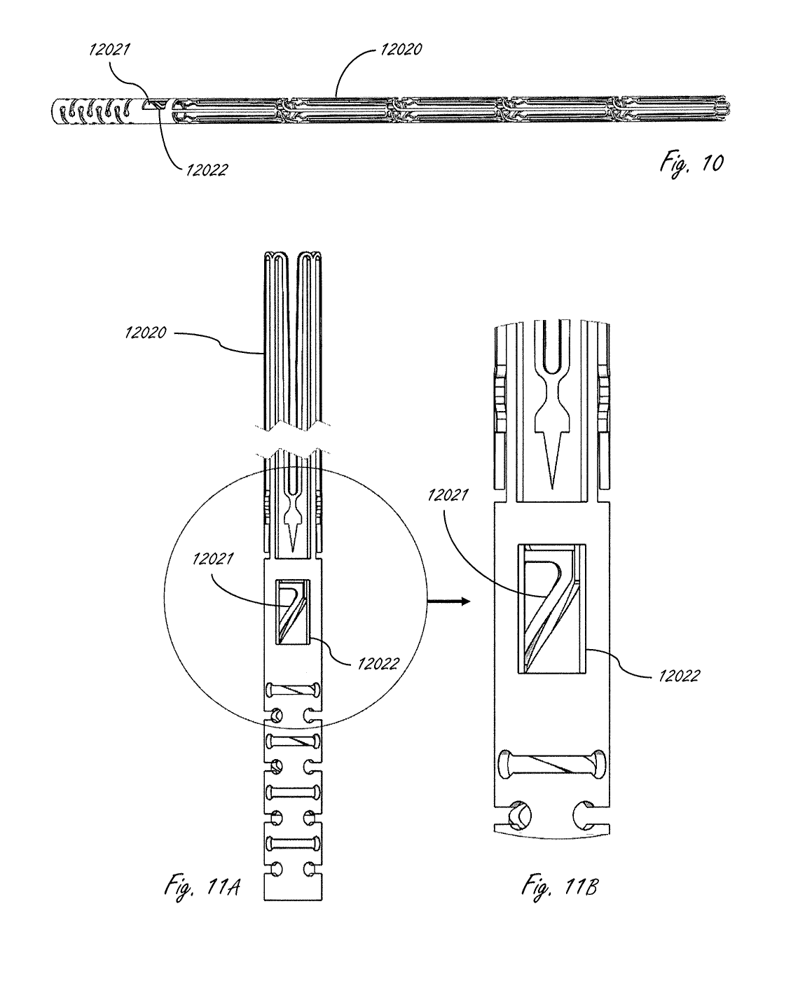

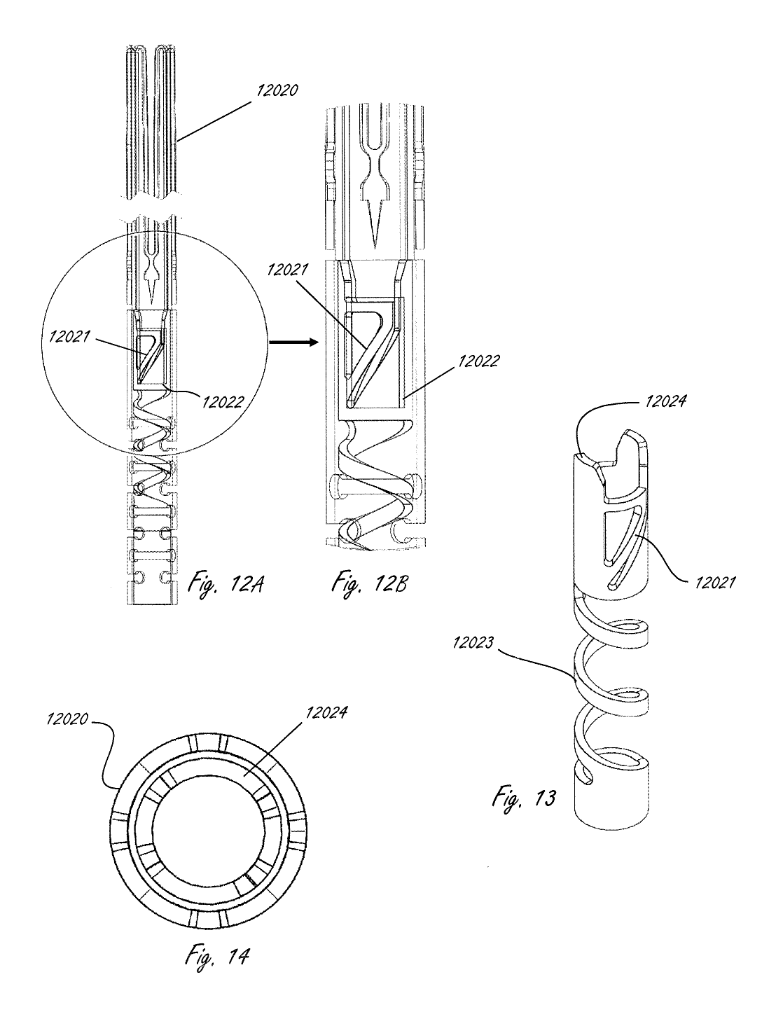

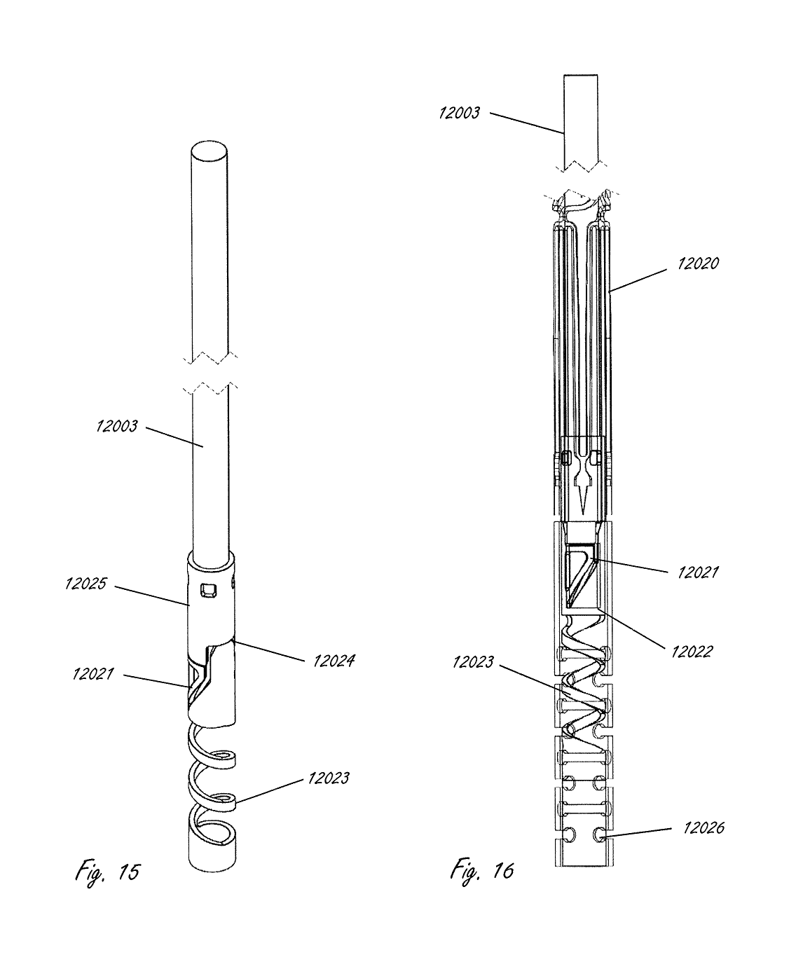

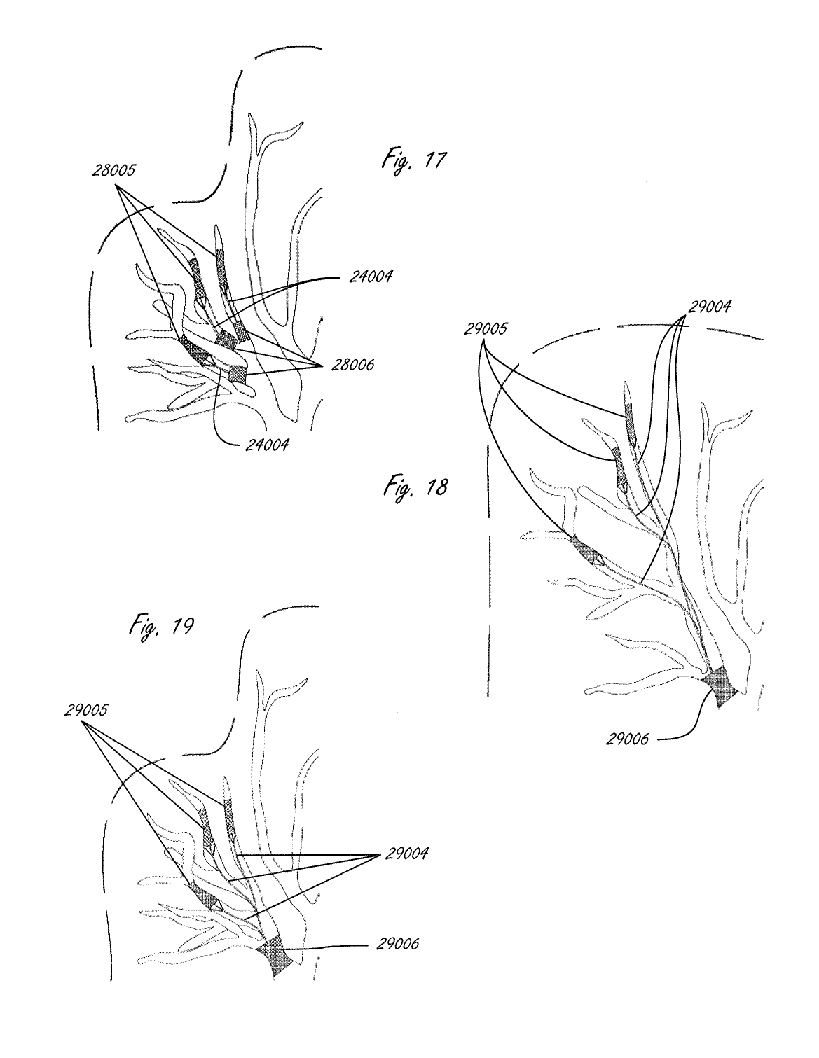

FIGS. 10 through 16 illustrate an exemplary mechanism configured to hold and adjust tension on a tether. This design includes a stent like tube (12020) shown in FIG. 12A, into which an expandable structure is cut. In one end of this tube a window (12022) is cut as illustrated in FIG. 12A and corresponding inset FIG. 12B. The spring like element of FIG. 13 (12023) is cut into a smaller tube that fits within the distal end of the larger tube 12020 and rests upon the flange of FIG. 16 (12026) fixed within the inner diameter of the outer tube. This flange prevents the element from moving beyond the tube under tension but allows for the element to rotate. A tab (12021) is cut into the smaller tube, which is then shape-set to extend slightly out of the surface of the inner tube and into that of the outer tube. The tab fits within the window of the outer tube. When the drive shaft of FIG. 15 (12003) with interface tip (12025) is advanced to the interface of the spring element (12024) and is rotated the element twists and the tab allows for rotational motion in only one direction. The orientation of the tab results in any rotational motion that is opposite of that which is desired being halted. This feature allows for tension to be increased or decreased and held in place. By rotating the tether it twists and foreshortens drawing the distal anchor it is affixed to in towards this proximal ratcheting structure. Should it be desired to release the tension in the line the drive shaft can be advanced further as to depress the spring of the spring element within the outer tube. When this spring is depressed the raised tab is forced to lay flat as it disengages from the outer tube window it extends into. As it is disengaged it is free to spin freely within the stationary outer tube. This design allows for a completely adjustable and reversible tension to be applied to tethers within the airways of the lung. FIG. 17 illustrates an embodiment of a method of reducing the volume of a lung by positioning a plurality of separated treatment devices within the lung. In this embodiment, each of the individual treatment devices includes a distal anchor (28005), a proximal anchor (28006), and a tether (28004), similar to the embodiment shown in FIGS. 1A-1C and FIG. 2. Each of the individual treatment devices can be actuated with a drive shaft to control the tension in the respective tether and thus the distance between the respective distal and proximal anchors. The physician may evaluate the resulting tissue response and may decide to continue treatment by increasing, decreasing, or maintaining the tension on each tether. The tension may be applied to all tethers uniformly, or may be applied individually depending on the adjustable proximal anchor design. Furthermore, the physician may choose to eliminate tension on the tether between anchors if it is no longer desired.

In some embodiments, a treatment device includes a plurality of distal anchors coupled to one proximal anchor. A tensioning component secured to the proximal anchor is actuated to modify the tension in the plurality of tethers. Each of the plurality of tethers can be individually tensioned or they can be tensioned together. The configuration of each of the tethers can thus be different, or the tethers can all change configurations to the same extent. FIG. 18 illustrates an exemplary embodiment in which the treatment device has been positioned within the lung and the plurality of distal anchors and the single proximal anchor are expanded and anchored to respective lumens. FIG. 19 illustrates the treatment device after each of the tethers has been tensioned, which has pulled each of the distal anchors towards the proximal anchor. In this exemplary embodiment, each tether is coupled to the proximal anchor at substantially the same location. In this embodiment of an adjustable anchor system for lung volume reduction, the apparatus includes a plurality of distal anchors (29005), an adjustable proximal anchor (29006), and tethers (29004) connecting the distal anchors and the adjustable proximal anchors. As previously discussed, the tethers may also be tightened in a stepwise fashion over time to provide maximal lung volume reduction while minimizing the chance of tearing of the parenchyma and other unwanted side effects (i.e. inflammation, bleeding etc.). FIG. 19 shows the apparatus after the tethers (29004) have been tightened and the delivery device removed. Because all of the anchors (29005) are tethered to a single adjustable proximal anchor (29006), they will all be drawn together towards a single location. The physician may evaluate the resulting tissue response and may decide to continue treatment by increasing, decreasing, or maintaining the tension on each tether. The tension may be applied to all tethers uniformly, or may be applied individually depending on the adjustable proximal anchor design. Furthermore, the physician may choose to eliminate tension on the tether between anchors if it is no longer desired.

The method shown in FIG. 17 may have an advantage of use when the lung tissues are more diseased and are not able to support the localized loading associated with a single location adjustable proximal anchor, such as in the embodiment shown in FIGS. 18 and 19. The method shown in FIG. 17 also allows the physician to only need to consider a single airway when placing each of the devices. Likewise, tensioning could be a simpler procedure because only one tensioning line is present in the airway, whereas the design shown in FIGS. 18 and 19 could require the user to discriminate between tensioning mechanisms for each tether. Alternatively, in some lungs there may not be enough healthy lumens in which to anchor more than one proximal anchor. In those situations, it may not be safe to use more than one anchoring device, each with its own proximal anchor. In these situations, a single proximal anchor design may provide the benefit of being able to be anchored in a single healthy tissue lumen while still being connected to a plurality of distal anchors. For example, in the embodiment in FIGS. 18 and 19, only proximal anchor (29006) need be anchored in healthy tissue. In FIG. 17, proximal anchor (28006) is anchored in healthy tissue. But if in FIG. 17 three healthy lumens cannot be detected, a choice of the procedure may be to use a single proximal anchor device.

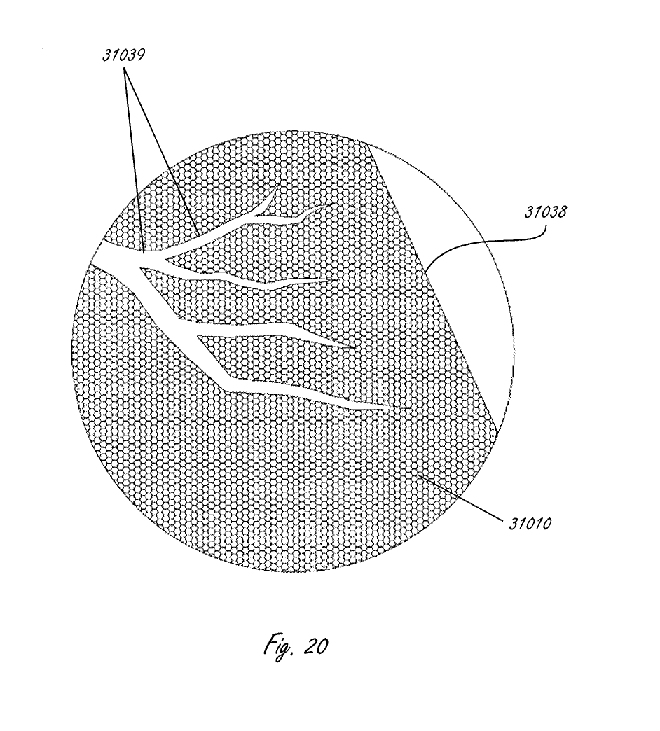

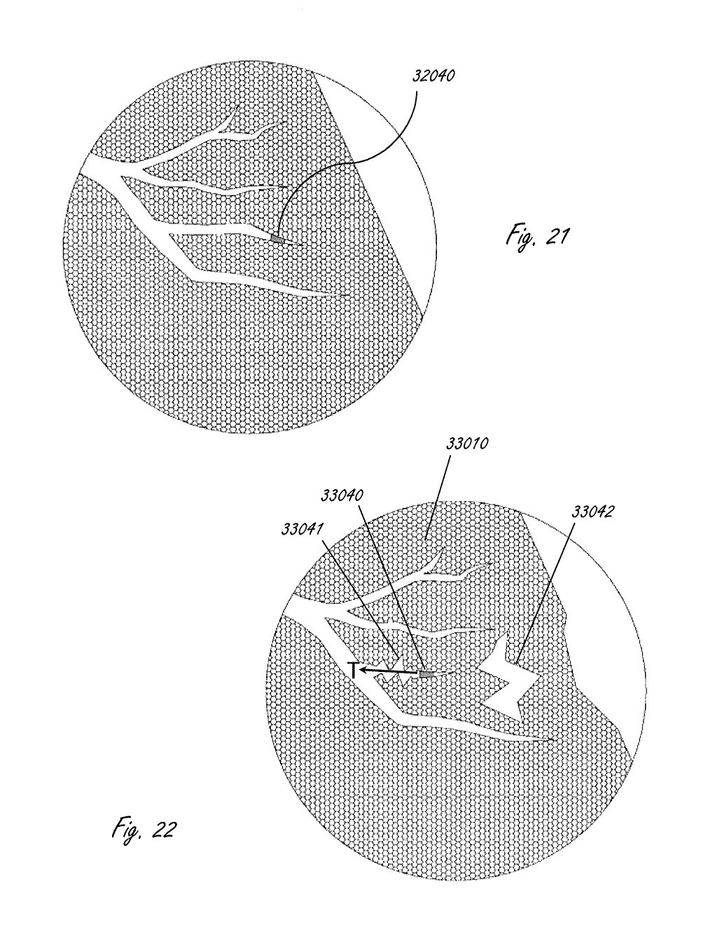

FIGS. 20-25 illustrate methods of use that can be used when placing a plurality of distal anchors in different lumens, regardless if one or more proximal anchors are used. Proximal anchors are thus not shown for clarity, but may be a single proximal anchor or a plurality of proximal anchors as described herein. FIG. 20 illustrates a sectional view of a portion of an emphysematous lung. FIG. 20 shows a surface of the lung, or visceral pleura (31038), a network of airways (31039), and a finer structure of bronchioles, blood vessels, and alveolar tissue herein referred to as the parenchyma (31010). FIG. 21 illustrates a single lung distal anchor (32040) configured for lung volume reduction. Tension (T) is applied to the anchor in FIG. 22 via a proximal anchor and tether (not shown for clarity), causing the adjacent airway (33041) to foreshorten. The tension is transmitted to the parenchyma (33010) surrounding the airway. The parenchyma (33010) is a delicate tissue, and in this case, the tension has exceeded the tensile strength of the parenchyma (33010), resulting in a tear (33042). The tear (33042) causes a degree of mechanical isolation between the airway containing the anchor and the outer extremities of the adjacent parenchyma, preventing the applied tension from reaching those extremities. As a result, the lung volume reduction is smaller than if no tear had occurred. Tearing is an undesired consequence and should be prevented.

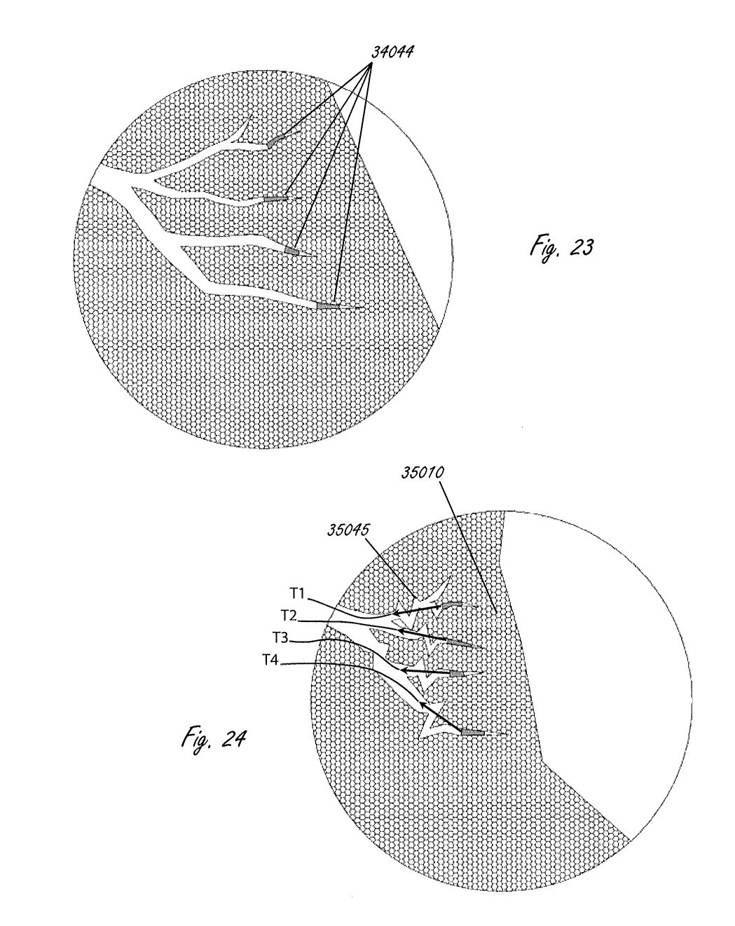

FIGS. 23 and 24 illustrate an exemplary embodiment of a method using a plurality of distal anchors for lung volume reduction. In this embodiment, a plurality of lung anchors (34044) are utilized. Tensions (T1, T2, T3, T4) are applied by tethers (see FIG. 24) interfacing either a single or multiple proximal anchors (not shown) causing the adjacent airways (35045) to foreshorten. The tensions are transmitted to the parenchyma (35010) area surrounding the airways (35045). While the parenchyma tissue (35010) remains delicate, the applied loads are spread over a larger area, and do not exceed the tensile strength of the parenchyma. As a result, no tear is formed, and the applied tensions can reach the outer extremities of the parenchyma. A much more effective lung volume reduction is achieved by avoiding tearing of the parenchymal tissue.

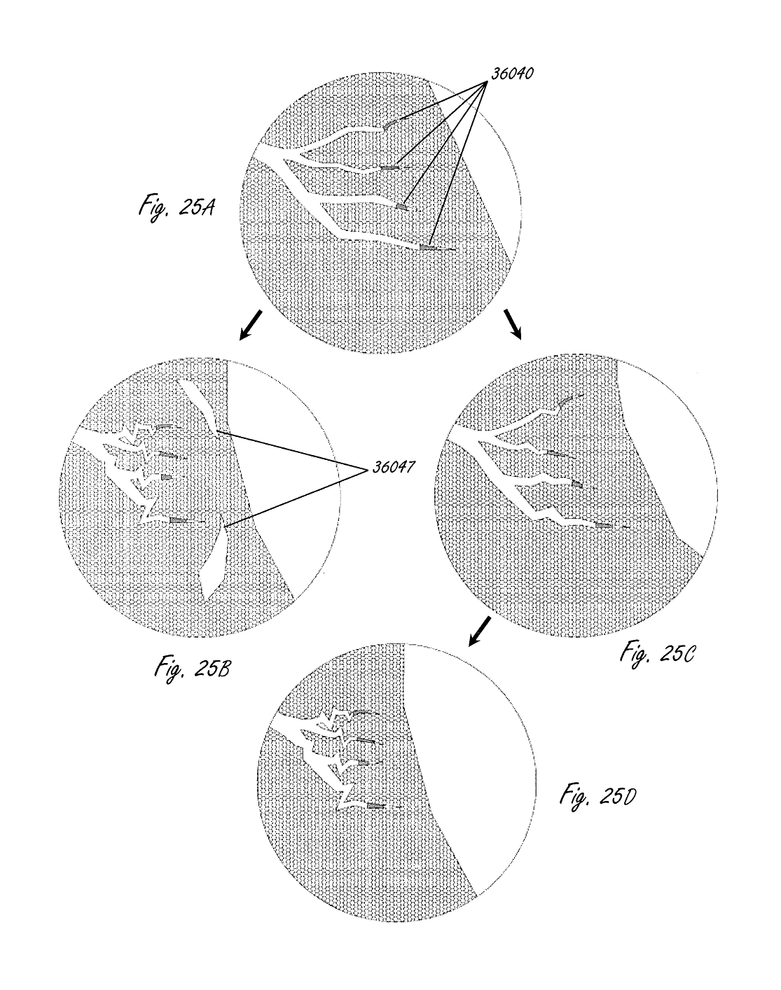

FIGS. 25A-25D illustrate an embodiment in which tension in respective tethers can be individually controlled. This embodiment also illustrates advantages of timing aspects of tensioning a plurality of tethers. FIG. 25A illustrates a portion of an emphysematous portion of the lung, wherein a plurality of lung anchors (36040) have been placed. FIG. 25B shows a potential result if a high level of tension is immediately placed on the anchors (36040). Tears (36047) are formed due to the high level of tension applied resulting in a reduced ability to reduce lung volume as similarly discussed for FIG. 22. Alternatively, FIGS. 25C and 25D illustrate a result if the tension to the tethers and anchors is applied stepwise and sequentially. An initial tension applied to all anchors as shown in FIG. 25C is significantly less than what will cause tearing in the parenchyma. After the initial tensioning, a period of time is allowed to elapse before applying additional tension. After the period of time has elapsed, additional tensioning is applied to all of the anchors, as shown in FIG. 25D. By performing the tensioning in a stepwise and sequential fashion, healing can occur in the tissue between tensioning events, which will allow greater ultimate deformation in the tissue without tearing. Another advantage of this stepwise tensioning is that any inflammation or other biological response from each tensioning event can subside before performing the next tensioning event. Additionally, imaging studies (e.g., X-ray, CT, MRI, and the like) may be performed between tensioning events to evaluate the impact of the previous tensioning event, and provide guidance for further tensioning events. Varying levels of tension may be applied to each anchor in order to maximize its reduction in lung volume, while preventing tearing of the parenchyma. In some situations, it will be appropriate to perform the procedure in either a stepwise or a sequential fashion. In some embodiments in which stepwise and sequential tensioning are performed, one proximal anchor is used, and in some embodiments a plurality of proximal anchors are used.

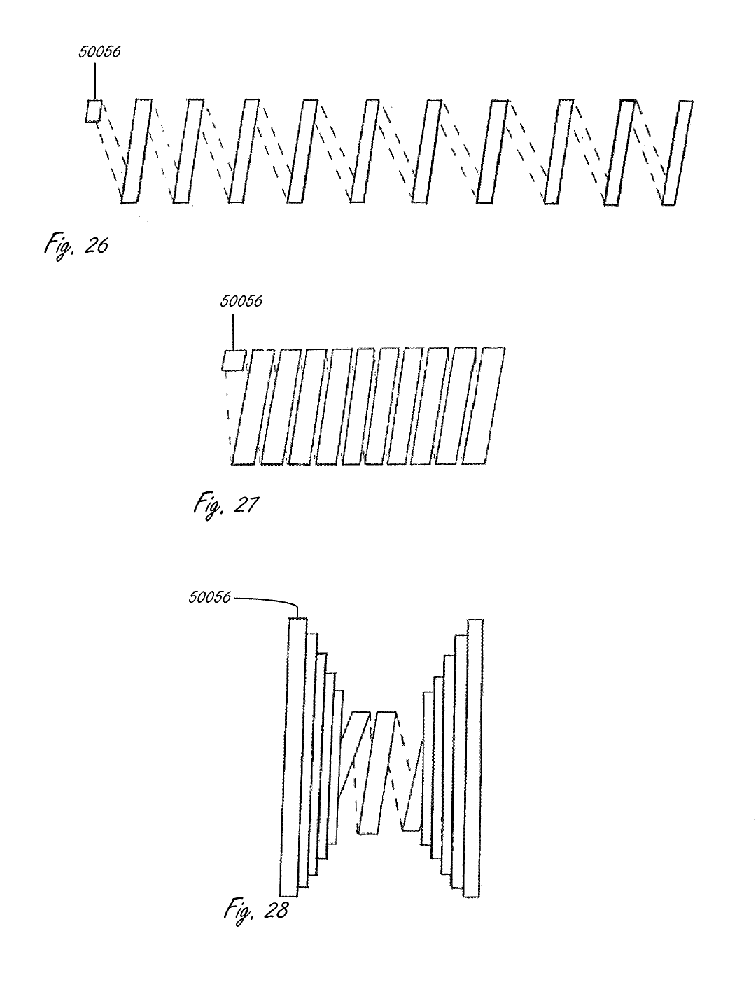

In some embodiments, the tether comprises a spring or spring-like element (generally referred to herein as a “spring”). The spring can be stretched to an extended length, as shown in the exemplary embodiment in FIG. 26, and released. This device could be shape-set to have a relaxed state resembling that of a helix, such as is as shown in FIG. 27, where the edge of each element (50056) comes into contact with itself along the trailing edge as it wraps around the spiral path. The device could also be set to resemble a torsional spring, such as in the exemplary embodiment shown in FIG. 28. In this torsional spring configuration, the longitudinal elements on opposing ends of the device lay over one another as they wrap in ever-increasing diameters. While the helical and torsional spring designs could be stretched prior to delivery and appear to have the same pattern, when the two designs relax the amount they foreshorten, as well as radially expand, will vary.

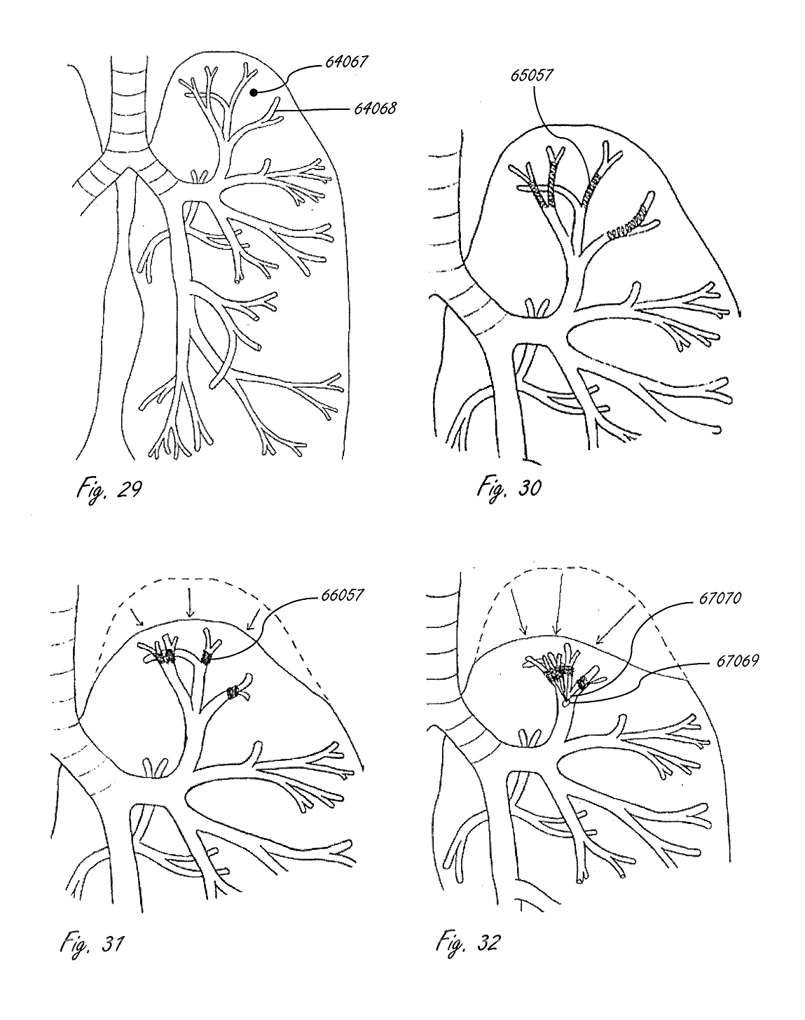

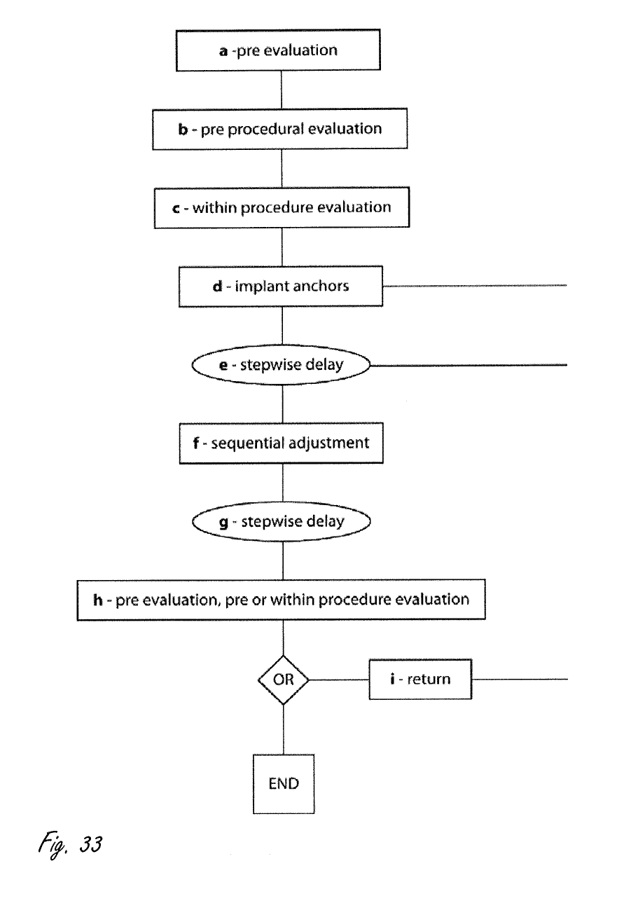

FIGS. 29-32 describe alternative methods and devices for lung volume reduction. FIG. 29 shows an illustration of a hypothetical human lung for a patient suffering from emphysema. The hypothetical target tissue for volume reduction (64067) is identified in the left superior lobe, or upper right of the illustration. Each device is individually introduced into the desired airway (64068) of the lung. A single device (65057) can be delivered to a single airway or multiple devices to several airways, as seen in FIG. 30. The device is released, and as it foreshortens from the spring force it draws in the engaged tissue, reducing the volume of the tissue attached to the airway in that portion of the lung, as seen in FIG. 31. The devices can stand alone as a unitary feature, or can be connected to a central node (67069) at a bifurcation via anchoring lines (67070) as shown in FIG. 32. Should anchoring lines be drawn to a node, a tethering system could be used to fix the lines and hold them in place. This system could allow for adjustability through the ability to individually change the tension on each of the anchoring lines. Each device is removable, as is any node or anchoring line that may be added as an option. The devices (65057) may be comprised of a super elastic material such as but not limited to memory metals. Additionally, in some configurations the elements (65057) may rely on the memory characteristics to transform from a delivery to a delivered configuration at implant. In particular as shown the device (65057) can be delivered at a temperature lower than body temperature, and rely on body heat to bring about a transition into the compressed state. Alternatively, the design can comprise a transition temperature greater then body temperature and rely on heating the device (65057) after delivery using the delivery tool, either by direct heating or joule heating mediated by inductive coupling.

FIG. 33 presents an exemplary flow chart of possible steps for use in performing a lung reduction volume method, examples of which are described herein. Not all steps need to be performed, and the order or steps can be modified if desired. A pre-evaluation step comprising imaging and or functional tests as described above is performed. Target and/or probable target tissues are identified at this stage. Next, a pre-procedure evaluation may be performed using minimally invasive techniques such as intra-bronchial ultrasound, local intra bronchial ventilation measurements, other characterizations of tissue density or compliance, or any pre-evaluation technique. The next step is to implant the anchors. At this point an optional stepwise delay may be initiated to allow for a healing response, tissue relaxation, and/or ingrowth. Next, a sequential adjustment is performed. This can be followed with a repeat evaluation chosen from any or any combination of those previously described. At this point additional anchors may be desired and the procedure is re-entered at step “d,” an additional stepwise delay may be initiated and the procedure re-entered at step “e,” or the procedure may be considered complete.

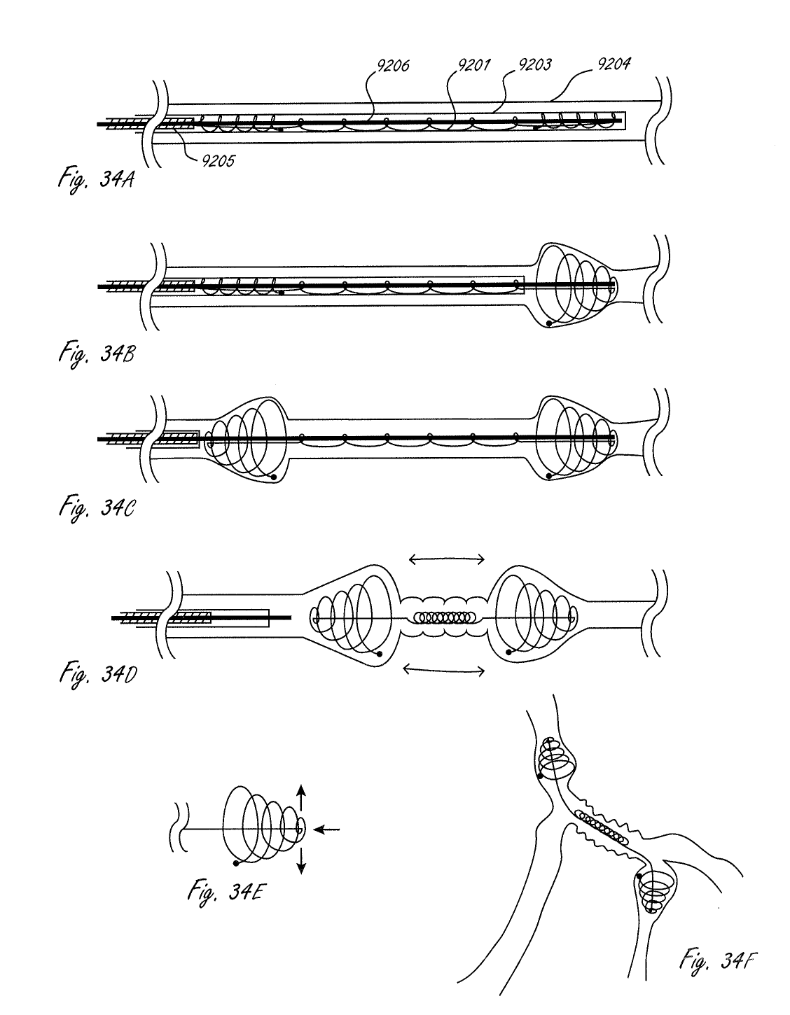

The examples shown and described with respect to FIGS. 34A-42E below build on the disclosure above, and any aspect of the systems and methods above can be incorporated into any of the examples in FIGS. 34A-42E unless indicated to the contrary.

FIGS. 34A through 42E illustrate additional non-traumatic anchors for lung volume systems, devices, and methods of use. “Non-traumatic” as used in this context refers generally to anchors that are designed, adapted, and configured such that no portion breaks through the bronchi with which it contacts. Such devices are adapted and configured to anchor by significantly expanding the bronchi, generally by at least 50% up to 500% or 1000% while not rupturing the bronchi. FIGS. 34A-34F depict an embodiment and deployment sequence for an anchor similar to that described in FIG. 28. In this embodiment, the spring element is wound such that the conical end sections have a relaxed configuration in which the apex of the conical section is at the longitudinal extreme of the spring. Such an embodiment allows for a more abrupt transition in bronchi diameter in the direction of the applied tension. In FIG. 34A the implant spring 9201 is contained within a delivery catheter 9203 in a delivery configuration. Also comprised in the delivery apparatus are a proximally attached pusher tube 9205 and a distally attached pusher rod 9206. On delivery, the pusher rod and tube are initially moved in unison thereby holding the spring in an elongate configuration. FIG. 34B illustrates the implant 9201 after the distal end has been released from the containment within the delivery catheter and the distal end has expanded into the bronchi 9204 thereby expanding bronchi. In FIG. 34C, the spring has been completely released from within the delivery catheter 9203, while the proximal to distal distance of the spring is maintained by the pusher tube and pusher rod, and proximal and distal ends have expanded into and anchored on the surrounding bronchi 9204. FIG. 34D illustrates the bronchi, axially compressed by the spring, after the distance between the distal ends of the pusher rod and tube are no longer constrained. Note that foreshortening of the spring as shown in FIG. 34D relative to FIG. 34C causes the distal expanded sections to collapse on themselves and future support the anchoring effect as noted in FIG. 34E where the arrows indicate the expansion of the anchor as an extreme end is pulled towards the center of the spring. FIG. 34F illustrates such a spring device in a delivered (foreshortened) configuration spanning a few branches of the brachial tree.

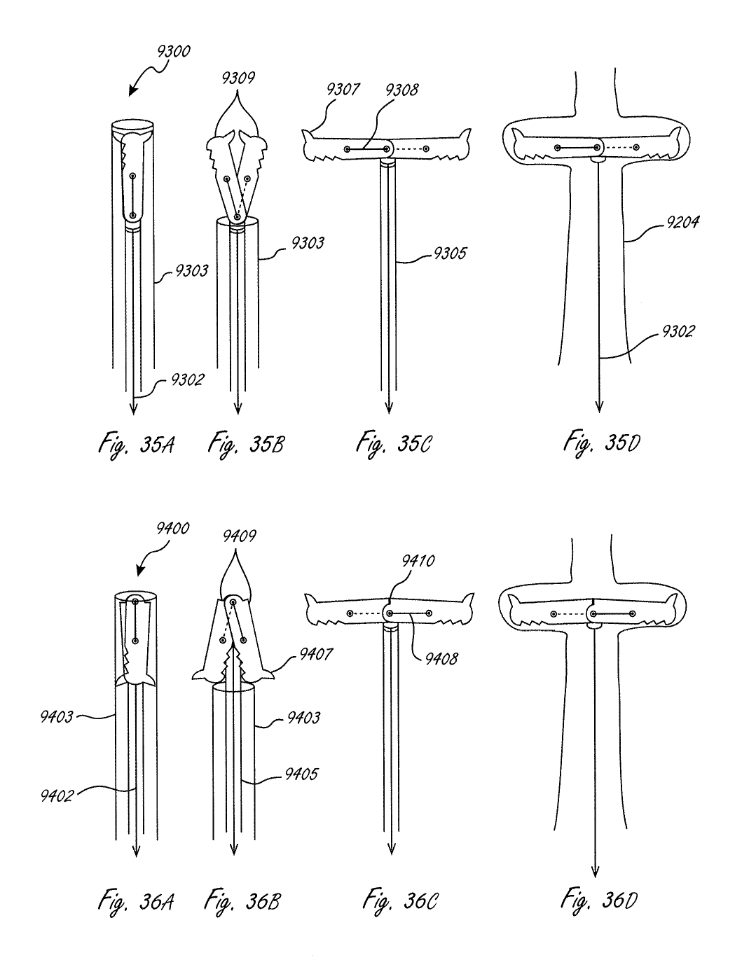

The examples in FIGS. 35 through 42 describe alternate embodiments for anchoring systems comprised of two or more distal anchors and a common pull wire proximal junction, where the proximal pull wire junction provides the function of a proximal anchor. The proximal anchoring function derives from the fact that the junction sits at a bifurcation of bronchi.

An unexpected result of experimenting and testing was the observation that a bronchial biopsy forceps, delivered via a bronchoscope, was very difficult to remove after it was opened/expanded. It was unexpectedly further observed that an anchor with a similar design and/or function could act as a distal anchor and provide adequate anchoring. FIGS. 35A, and 36B-C illustrate additional embodiments for anchoring structures which open around a central pivot to expand in a scissoring fashion. These anchors are comprised of a pull wire and an expanding anchor structure comprised of scissoring jaws which optionally comprises any or any combination of the following; a catch or stop which constrains the anchor from opening past a maximum rotation; a spring to aid in the opening after release from the delivery catheter; and barb structures which aid in the opening of the anchor by allowing the outer ends of the opening structure to engage bronchial tissue and thereby limit the anchors' ability to slip along the bronchi when pushed or pulled during deployment. As illustrated, each is allowed to rotate through 90 degrees and forms a linear structure in the fully deployed (which may also be referred to herein as fully expanded) configuration. The delivery structures for these embodiments comprise a delivery catheter and a means to push the anchor out of the delivery catheter.

FIG. 35A illustrates an anchor and deployment system which opens by scissoring towards the delivery tube. As illustrated the anchor comprises scissoring jaws 9309, and pull wire 9302, optional barbs 9307 and spring(s) 9308. The spring in some embodiments will be capable of fully deploying the anchor and in other embodiments the spring action may be supplemented by the action of the barbs which on engagement with the tissue allow the user to push on the center of the anchor until the anchor stop is engaged to complete the opening. FIGS. 36B and 36C illustrate an anchor and deployment system which opens by scissoring away from the delivery tube. As illustrated, the embodiment comprises scissoring jaws 9409 and pull wire 9402, optional barbs 9407 and spring(s) 9408. The spring in some embodiments will be capable of fully deploying the anchor. In other embodiments, the spring action may be supplemented by any of the action of the barbs, which upon engagement with the tissue allow the user to pull on the center of the anchor via the pull wire 9402 until the anchor stop 9410 is engaged to complete the opening, and/or after the anchor is pushed out of the delivery catheter the anchor may be pulled against the delivery catheter with the pull wire 9402 thereby fully opening the anchor. FIG. 36C illustrates the anchor from either 35A or 36B in a fully deployed configuration within a bronchi, illustrating the anchoring of the anchor and the reconfiguring of the bronchi by the anchor.

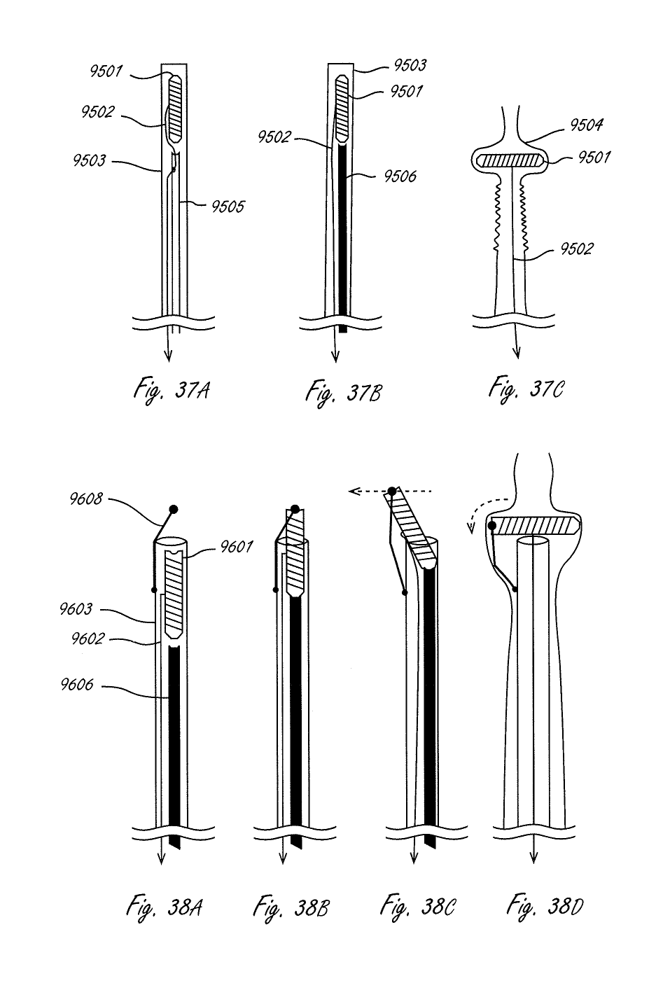

FIGS. 37A-C illustrate an exemplary non-traumatic distal anchor 9501 connected, either directly or indirectly, to a pull wire or tether 9502 that anchors by rotating 90 degrees to the bronchi axis on delivery, thereby expanding a section of the bronchi. FIGS. 37A and 37B illustrate two alternative deliver configurations for such a device. Each delivery configuration comprises a delivery catheter 9503 which may be comprised of the working channel of a bronchoscope or may be a separate catheter, an anchor 9501 attached directly or indirectly to a pull wire 9502, and a delivery tool comprising a push tube 9505 or a push rod 9506. In FIG. 37A the delivery tool push tube 9505 comprises a rapid exchange feature (which may be incorporated in any of the designs disclosed herein which use a push tube) through which the pull wire 9502 is run, in an alternate configuration the pull wire maybe run through the entirety of the push tube. Deployment is accomplished by pushing the anchor out of the delivery catheter with the push tool and then pulling the anchor back against the delivery catheter with the pull wire to force the anchor to rotate with respect to the delivery catheter. The delivery catheter is then removed, leaving the anchor in the rotated and anchored position and configuration. FIG. 37C illustrates anchor 9501 (from either FIG. 37A or 37B) in a fully deployed configuration. In such an embodiment, the anchor may comprise a barb as discussed in conjunction with the embodiments illustrated in FIGS. 93 and 94 to help facilitate the rotation. FIGS. 38A-D depict an alternative delivery mechanism for an anchor similar to that of FIGS. 37A-C in which rotation of the anchor 9601 is facilitated by spring structure 9604 affixed to the side of the delivery catheter, 9603. As the anchor is pushed out of the delivery catheter the distal end of the anchor engages with the rotation spring. As the anchor is pushed further it is forced to rotate, with the distal end rotating to the left in the figures.

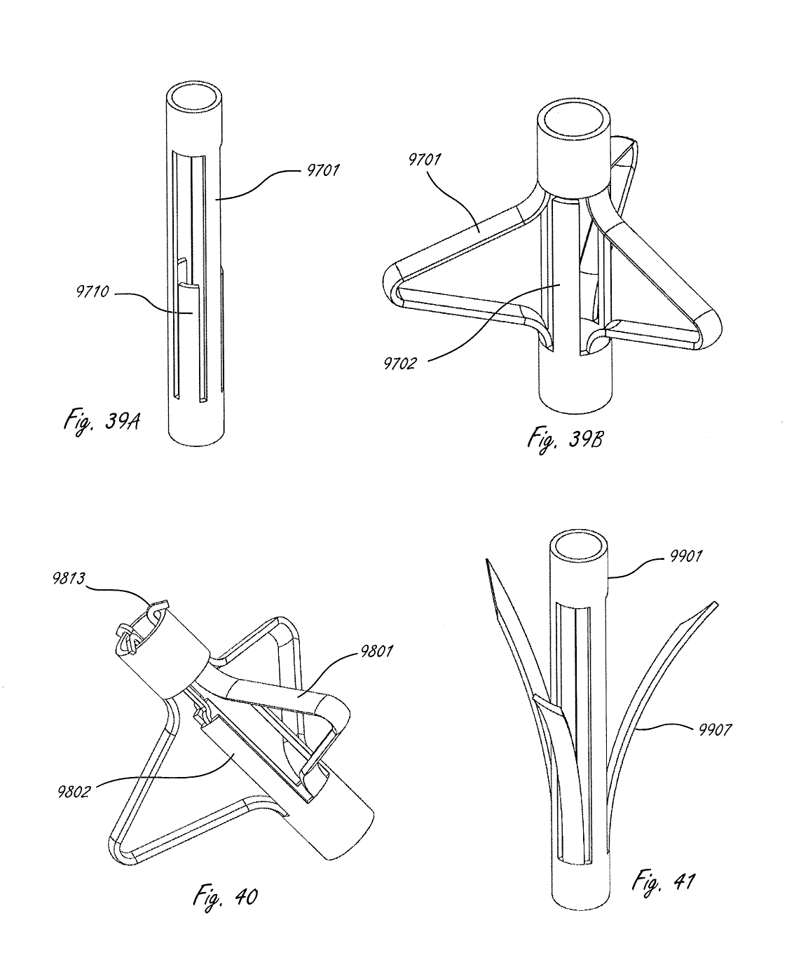

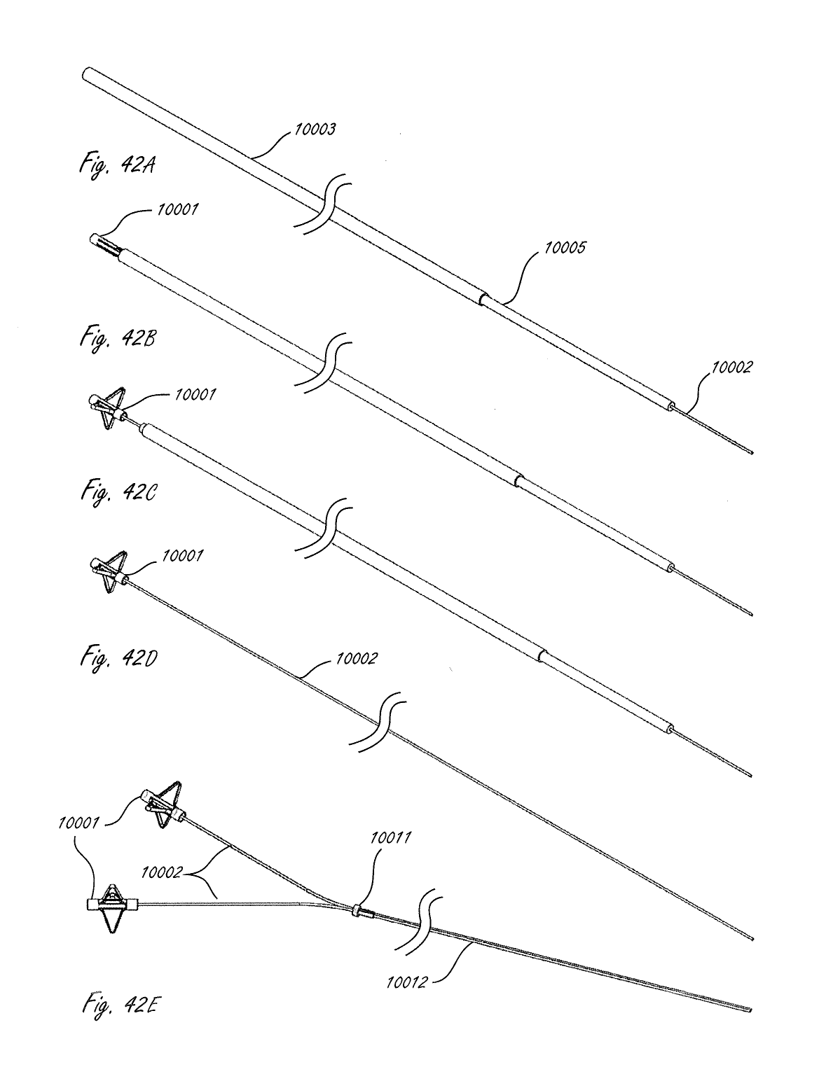

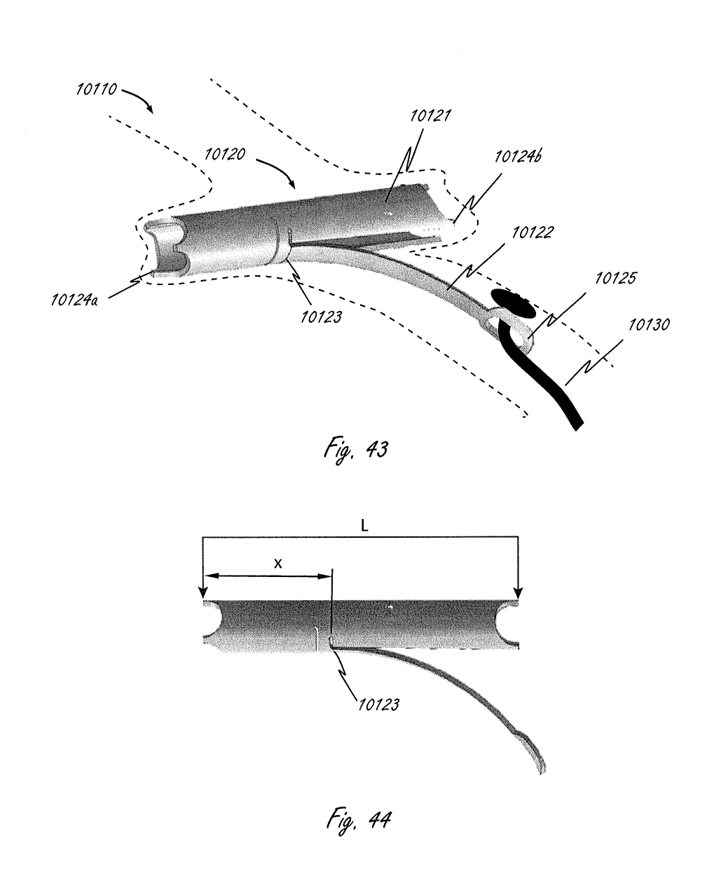

FIGS. 39 through 41 illustrate three additional configurations for distal anchors, two of which are non-traumatic (FIGS. 39A/B and FIG. 40) and one is traumatic (FIG. 41). Each anchor would be affixed to a pull wire attached at the distal end, which is facing the bottom of the page for each of the embodiments illustrated.

FIGS. 39A and 39B illustrate an expandable anchor 9701 which may be cut from a nitinol tube or similar material prior to shape setting as illustrated in FIG. 39B. The anchor can be collapsed for introduction into a deployment catheter. Anchor 9701 also comprises an optional deployment stop 9710. Such an anchor may in some embodiments be capable of full deployment as facilitated by the spring forces inherent in the shape set structure. Alternate embodiments may require additional force to deploy, such as by pushing a deployment tool against the proximal edge of the anchor while constraining any displacement of the distal end with the distally affixed pull wire. The deployment stop 9702 assures that anchor is not over-compressed in such a deployment.

FIG. 40 illustrates an anchor 9801 similar to that of FIGS. 39A-B in the shape set configuration. The anchor 9801 additionally comprises a plurality of clips 9813, cut as part of the structure, which engage the proximal end of the structure on deployment. This engagement locks the structure in a deployed configuration and elements 9802 constrain the anchor from over compression.

FIG. 41 illustrates a traumatic variation in which the barbs 9907 may be laser cut from a nitinol or similar material tube.

FIGS. 42A-E illustrate an exemplary deployment sequence and a deployed configuration for the anchor of FIGS. 39A and 39B.

The anchor and deployment system 10000 comprises a delivery catheter 10003, a push tube 10005, and the anchor 10001 and associated pull wire 10002. During a deployment, the deployment or delivery system is loaded into the working channel of the bronchoscope and the bronchoscope delivered to a location in visual in contact with the target location. As an alternative, the bronchoscope may be delivered to the target location and then the delivery system loaded into the bronchoscope. Upon reaching a location in visual in contact with the target location the delivery system is pushed out of the distal end of the bronchoscope working channel and into the target bronchi. The push tube is then used to push the anchor out of the delivery catheter as illustrated in FIG. 42B. The anchor expands as it is released from the delivery tube. As indicated above in some embodiments the anchor may further expanded by constraining the distal end with the pull or anchor wire and pushing the proximal end with the push tube. This is especially useful when a self-locking anchor such as that depicted in FIG. 40 is used. The delivery components 10003 and 10005 are then removed as illustrated in FIG. 42D. At least one additional anchor, if not more, are then placed in the general vicinity in the same fashion. Upon completion of anchor placement, a clamping bead 10011 is slid down the bundled pull wires 10012. As illustrated the bundle comprises two pull wires, but as indicated above such a bundle may comprise more than two pull wires 10002. The clamp 10011 is pushed until its distal end encounters at least a bronchial bifurcation into each branch of which a pull wire in the bundle trails. The clamp interfering with this bifurcation comprises the proximal anchor in such a deployment.

In some embodiments, a clamp may be used in a similar fashion to gather multiple bundles of pull wires 10012, associated with already clamped and volume reduced regions, at a bifurcation more proximal than those associated with the gathered bundle to further compress lung tissue. This is especially useful when returning to further compress tissue as described in procedures elsewhere in this disclosure.

In practice multiple of such anchor placements may be used to achieve a complete LVR.

In some embodiments, the delivery catheter 10003 is short and serves to load the remaining portions of the system into the working channel of a bronchoscope and the anchor deployment occurs at a location just distal to the distal end of the bronchoscope.

In some embodiments, the delivery is done under fluoroscopic imaging with the aid of an external mapping system such as the CARTO or NaviStar systems without the use of a bronchoscope.

Any of the pull wires described herein, including in FIGS. 34-42, may be comprised of metals or polymers. In any of the examples the pull wire may be beaded or shaped in such a fashion that they interface with the clamp in the fashion of tie wraps.

In any of the examples in FIGS. 34-42, the width of the anchor in a deployed configuration (measured orthogonally relative to the longitudinal axis of delivery device) may be from 0.5 mm to 8 mm, and can be, for example, chosen from a kit of devices depending on the anchoring location. Some smaller lumens may need a smaller width anchor, while larger lumens may require larger dimensioned anchors for proper anchoring. Additionally, the aspect ratio of anchor width to height (height measured in the direction of the longitudinal axis of delivery device) may be 2/1 to 10/1, such as about 4/1-5/1.

Additionally, in the examples in FIGS. 35-38, the anchor is shown and described as linear. The anchor may have an expanded configuration that is not quite “T” shaped, but one that is closer to “Y” shaped. For example, in some embodiments the angle of the deployed anchor, relative to the longitudinal axis of the delivery device), can be from 90 to 135. In some embodiments it may also be slightly less than 90 degrees.

This disclosure incorporates by reference herein the disclosure of U.S. Pat. Nos. 6,997,189 and 8,282,660. Any of the embodiments therein can be modified to include any of the features or methods of use described herein.

As noted in FIGS. 35-38, distal anchors are deployed such that they form a general “T” shape after exiting the delivery tube of a delivery system. The devices are adapted and configured so that the top arms of the “T” (deployed approximately perpendicular to the longitudinal axis of the airway) are compressed or collapsed in the delivery tube until deployed out of the delivery tube. The designs of FIGS. 35-38 are not intended to necessarily puncture like a barb through the wall of the bronchi, but rather rotate within the bronchi, or otherwise deform the bronchi and surrounding tissue, such that the bulk of the anchor within the deformed bronchi space prevents the anchor from being pulled out under tension. Additional anchor concepts are described herein, including those of FIGS. 39-42. Alternative designs to accomplish the same or similar effect are described below that allow for delivery and deployment of a “T” shaped anchor (at least the formation of a T-shaped distal end in response to deployment of a portion of the anchor), as well as to help secure it in lung tissue while under tension. Other inventive ways of connecting and tensioning the distal anchor to the proximal anchor are also described.

In a specific but exemplary embodiment of FIG. 43, the distal anchor 10120 is formed from a tubular element 10121, and includes an extension 10122 (which can be a tether extension) that extends from the tubular element 10121 at extension location 10123. The ends of the tubular element 10121 can be formed to comprise one or more somewhat pointed edges 10124a, 10124b, capable of digging into, though not necessarily perforating, the wall of the bronchus. Edges 10124a, 10124b may not be pointed, but can protrude, from the ends of the tubular element 10121, optionally outward in a direction generally parallel with a longitudinal axis of element 10121, so they can effectively anchor against into the wall of the bronchus. A tension line 10130 (which can be a tether) is secured to the extension 10122 at location 10125. The location 10125 preferably extends beyond the end of the tubular element 10121 when compressed in a delivery system, such that any knot-like structure affixed against location 10125 is beyond the tubular element such that it has space to reside within the delivery system.

As illustrated in the exemplary FIG. 43 embodiment, the distal anchor, including the extension, is fabricated from a single tubular material (i.e., the extension is integral with the adjacent material of the tubular element). The extension can be formed by laser cutting the tubular material. The material may be nickel titanium (nitinol) and the part can be fabricated using a laser cutting or etching process. This allows the part to be formed from a single component without the need for a bond joint. The extension may be heat set (e.g., at 504° C. for 5 minutes) into a shape such as that illustrated where the extension has a configuration that is curved radially away from the tubular configuration of element 10121. This enables the tubular element 10121 to spring out and rotate in the lumen as it is deployed out of the delivery catheter. The strain and stiffness of the extension and tubular element may be optimized with the selection of the tube outer diameter and wall thickness, tube length, the width and length of the tether extension 10122, and the radius of the heat set curve of the tether extension. To minimize strain at the attachment point 10123, additional cuts in the tubing may be added for strain relief of the element. In the specific embodiment of a nitinol tube, the tube may have an OD of 0.050″, wall thickness of 0.005″, and total length of 0.276″. In a merely exemplary embodiment, the extension may have a length of about 0.276″, width of about 0.30″-about 0.20″, and heat set radius of about 0.138″. All of these values are approximate and could be easily varied ±50% to achieved desired results.

In alternative embodiments, any of the extensions herein can be a separate component that is attached to the rest of the anchor, and thus need not be integral with the rest of the anchor.

The tension tether line 10130 may be constructed from a monofilament or braid of a suitable high strength flexible polymer such as nylon, polypropylene, polyester or silk (round or flattened cross-section). The material may also be a strand or braid of round or flattened metal wire, such as stainless steel or nitinol. The line could alternatively or additionally be fabricated from biocompatible radiopaque materials such as metals (platinum, tungsten, tantalum, etc.) or polymers containing radiopaque additives (e.g., powders of barium sulfate, bismuth subcarbonate, tungsten, etc.). The line may be fabricated with features to encourage its grip in a proximal ratchet feature using a process such as extrusion, molding, insert molding, die cutting, laser cutting, or any combination of these. The line 10130 may be secured to the distal anchor 10120 by tying it in a knot around a suitable feature in the anchor such as the loop or hole formed at location 10125 in FIG. 43. The fixation may also be simply accomplished by using an enlarged feature such as a knot tied to be larger than the hole through which the line passes, such that the knot cannot pass through the loop/hole at location 10125 under tension. The knot could be held in place with a heat setting and/or molding process, or using an adhesive. The enlarged feature could also be adhesive itself or an insert molded material around the end of the line or around a knot in or adhesive around the line. The line could also be passed through a hole in a bead-like element and secured to itself. In the case of a line constructed from metal, an enlarged ball may be formed on one end (e.g., using an arc welding process), or a metallic tube crimped to the end of the line, to achieve the same result of the knot described above.

As illustrated in FIG. 44, the attachment location 10123 may be a distance x from one end, which is less than the total length L. In the illustrated embodiment, the attachment point is a distance x where x is approximately (⅓)*L, but can also be other fractions of the total length L. This distance may be optimized to optimize the ability of the tubular element 10121 to flip and secure itself against the wall of the bronchus. In some embodiments, values of x/L may range from approximately 0.10 to 0.50. Smaller values of x may increase the torque of the element and increase the force of the opposite end against the wall as the element rotates, while a value of about 0.5 would help balance the load between the two ends of the element.

In other embodiments related to FIG. 43 above, and FIGS. 37-38, the tension tether line 10130 may be attached directly to the tubular element 10121 without the need for a tether extension 10122. FIG. 45 illustrates the line 10330 passing through a hole or similar feature cut directly into tubular element 10321. An enlarged distal feature, as previously described, would prevent the line from pulling through the hole in the tube. As described above, the distal end of the tension tether line may have an enlarged portion (e.g., knot, welded ball, a bead through which the line passes, etc.) which resides within the tube while the remainder of the line passes through a hole or slot in the tube. FIG. 46 illustrates how during fabrication, the enlarged feature could be passed into the tube via a slot in the tube, and then the end of the slot constrained to prevent the line from coming out. FIG. 46 illustrates how this could be accomplished by crimping an edge of the tube while forming a groove for the line. The constraint could be any number of other methods such as applying adhesive to the end of the tube, bonding a ring or other element across the tube slot, or filling the entire tube with an adhesive or polymer to secure the line. The tube, if constructed from a thermoplastic polymer, could also be reshaped with heat to secure, or even weld directly to, the line.

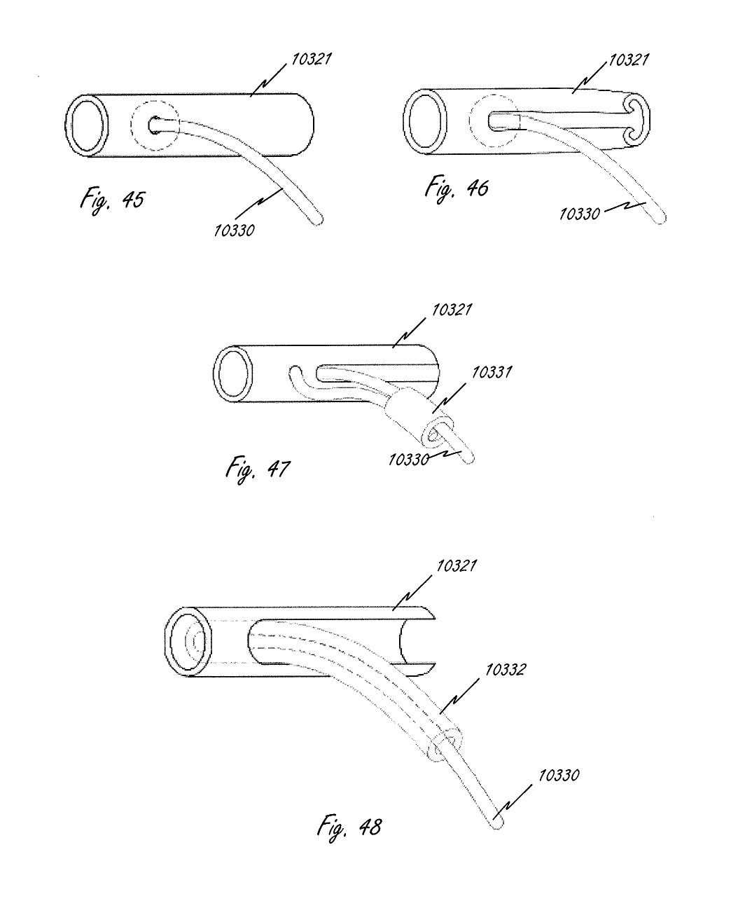

FIG. 47 illustrates how the line 10330 could also be looped through a hole or slot and another slot in the tubular element 10321 and bonded to itself (such as a heat weld of a polymer or an arc weld of a metal). Securing the line to itself can also be facilitated with a tube 10331 passing over the two portions of line to be bonded (as illustrated in FIG. 47). The tube 10331 could be used to contain an adhesive or be used to crimp or heat shrink over the two lines.

The distal anchor 10321 may also be formed from a solid component, rather than a tubular element. The tension tether line 10330 may be passed through a transverse hole in the solid component, similar to that shown passing through the tube in FIG. 45. The hole could be larger on the distal end than the proximal end to allow the enlarged element to recess within the hole. Additional longitudinal slots in the solid component could be made to recess the line while in the delivery system.

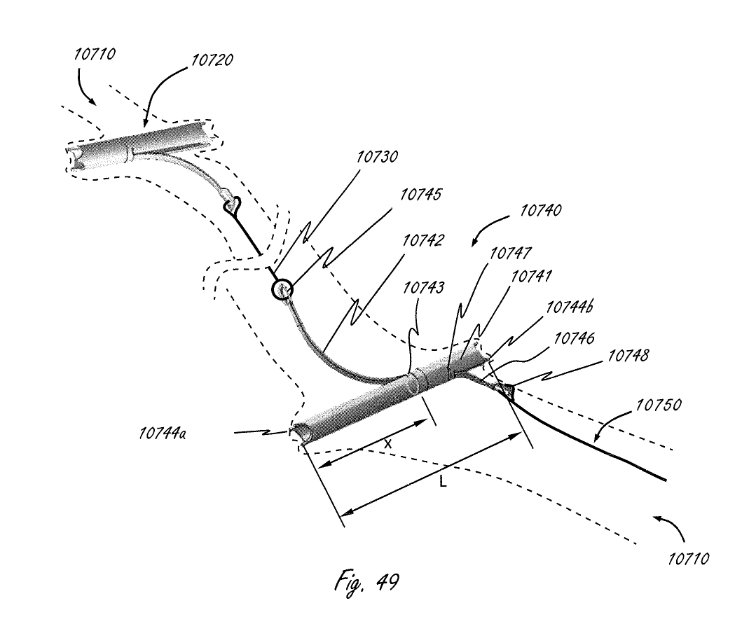

FIG. 48 illustrates how the tether extension 10322 shown in FIG. 43 could be a separate deflector 10332 attached to the tubular element 10321. The deflector is preferably a shaped spring-like component that is constrained straight in the delivery system but then deflects away from the longitudinal axis of the tubular element 10321 to encourage it to deflect in a direction generally more perpendicular to the bronchial lumen 10310 than when in the delivery system. The tension tether line 10330 is preferably directed within the deflector such that it is biased in the same direction as the deflector. In addition to deflecting the line 10330, the deflector 10332 may also serve to strain relieve the transition between the line and tubular element to reduce the chance of fatiguing and fracturing. The deflector could be a tubular structure (as shown), or a tube with slots of various orientations and separation distances to encourage flexibility and strain relief. It could also be formed of a coil or braid or any combination of these. Heat set polymers, nitinol, or stainless steel could be used to provide the spring shape. The distal end of the deflector 10332 could be secured to the tubular element 10321 by any of the ways previously described.

Instead of being fabricated from a metal such as nitinol, the distal anchor could also be fabricated from a polymer or polymer blend. Such polymers include, but are not limited to nylon, polyester, polyether, polypropylene, polyamide, polyimide, polyethylene, and PEEK. The anchor could also be a composite, such as a polymer molded over a metal or another polymer. To aid in fluoroscopic visualization, the anchor could include any number of biocompatible radiopaque materials such as metals (platinum, tungsten, tantalum, etc.) or polymers containing radiopaque additives (e.g., powders of barium sulfate, bismuth subcarbonate, tungsten, etc.). The distal anchor may also contain a coating to improve acceptance by the body. The coating may be antimicrobial coating known in the art to inhibit bacterial growth around or inside the implant. The coating may also or instead contain a drug to either accelerate/encourage tissue ingrowth by the surrounding tissues. To facilitate tissue ingrowth, the distal anchor 10321 (or any distal anchor herein), particularly the tubular element 10322, may have a surface which is at least partially porous, such as formed by many holes or slots. The tubular element could also be formed as a coil or a braid to allow for tissue ingrowth. A porous material covering such as ePTFE or a polymer mesh could also be used. The coating may also be used to delay tissue ingrowth. Delaying tissue in growth may serve to increase the time over which a physician may choose to remove the implant if the desired clinical effect is not being achieved, the patient is having exacerbation of symptoms, or a technical issue arises with the implant.

The anchors described above and many described herein are designed to be deployed as the distal anchor, with the tension tether line extending proximally to a proximal anchor. Various embodiments of the proximal anchor are described below.

In a relatively simple embodiment illustrated in FIG. 49, the proximal anchor 10740 may be designed and function much like the distal anchor 10720 described above, with the tension tether line 10730 attached to and extending between the two anchors. In the specific embodiment of FIG. 49, the proximal anchor 10740 is formed from a tubular element 10741. An extension 10742 extends radially away from the tubular element 10741 at extension location 10743. The ends of the tubular element are preferably formed into somewhat pointed edges 10744a, 10744b, capable of digging into, though not necessarily perforating, the wall of the bronchus (as described above). The tension tether line 10730 is secured to the tether extension 10742 at location 10745, which in this embodiment has a loop shape. The location 10745 preferably extends beyond the end of the tubular element 10745 when compressed in a delivery system, such that any knot-like structure affixed against location 10745 is beyond the tubular element such that it has space to reside within the delivery system. The location 10745 can have x/L values as described for the distal anchor.

As also illustrated in FIG. 49, the proximal anchor also has a proximal extension 10746 integrated into tubular element 10741 and extends radially away from the tubular element at extension location 10747. This feature is provided to allow the proximal anchor to be releasably secured at location 10748, which in this embodiment has a loop shape. The way of securing to the proximal extension may be through the use of a flexible polymer or metal wire line similar to that described for tension tether line 10730, or it may be grasped using conventional biopsy or grasping tools capable of being deployed through the working channel of an endoscope such as a bronchoscope. The extension feature is preferably heat set to deflect away from the longitudinal axis of the tubular element 10741 after deployed from the delivery system, and may have one or more bends therein. The way of doing this could be similar to that described for the extension 10722 and 10742. This would make re-grasping the feature easier after deployment in the airway lumen. The feature at location 10748 could be a loop/hole or an enlarged ball to enable grasping. While described as integrated into the extension 10746, the feature at location 10748 could alternatively be directly integrated into the proximal end of the tubular element 10741.

The alternate embodiments (e.g., features, materials, processes) described above for the distal anchor 10720 are also applicable to the proximal anchor 10740.

To facilitate anchoring in the larger diameter proximal airways, the length of the “T” style proximal anchor described above could be made longer than the distal anchor. In an example, the length L of the tubular element of the proximal anchor may be about 0.50″ with other dimensions similar to that of the distal anchor in FIG. 43. Similarly, the outer diameter of an expanded anchor such as that of FIGS. 39-42 could be made larger for the proximal compared to the distal anchor. In general, the length or outer diameter of either the distal or proximal anchor could be tailored to be oversized to the luminal diameter in the target anatomy. In some embodiments, the proximal anchor is at least 1.1-3 times the length of the distal anchor, such as 1.1-2.5 times.

To deliver the tethered distal and proximal anchors, the distal anchor could be loaded from the proximal end of the delivery system, followed by the tether, and then the proximal anchor. To advance the distal anchor out of the distal end of the delivery tube, a pusher tube advanced over a tensile line 10750 could be used to push against the proximal end of the proximal anchor to transfer the force to the distal anchor. The tensile tether line 10730 can be coiled within the delivery tube to facilitate transfer of the deployment force from the proximal to distal anchor. The tether line 10730 could alternatively be formed from a braid of wires and/or polymer strands, preferably with a central lumen. The braid may also have a polymer as a coating and/or embedded matrix. The line may or may not be heat set in the coiled or braided shape. Advancement of the distal anchor, tether line, and proximal anchor may be facilitated by the use of a guidewire, or similarly sized wire or beading, which passes through the center of the tubular elements and coiled tether line, and pusher tube, extending back out through the proximal end of the delivery system. In this embodiment, the guidewire may be in the same lumen as the tensile line 10750, or the pusher tube could have separate lumens for the guidewire and tensile line. As previously described, the pusher tube could be configured to be a “rapid exchange” style tube for one or more of the lumens described. Alternative to the use of the pusher tube and line 10750 would be to attach a grasping mechanism to the proximal end of the proximal anchor (preferably at location 10748 of extension 10746) and use that to push forward the distal anchor.

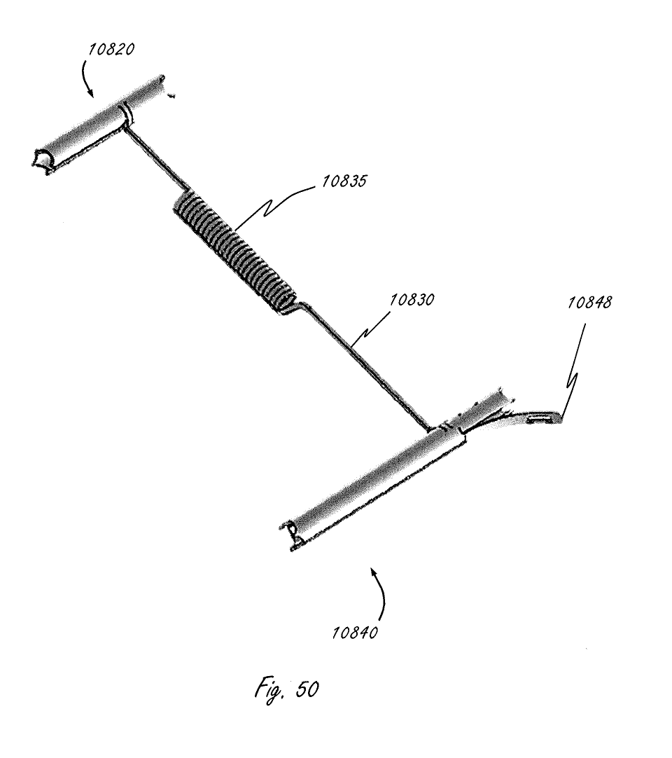

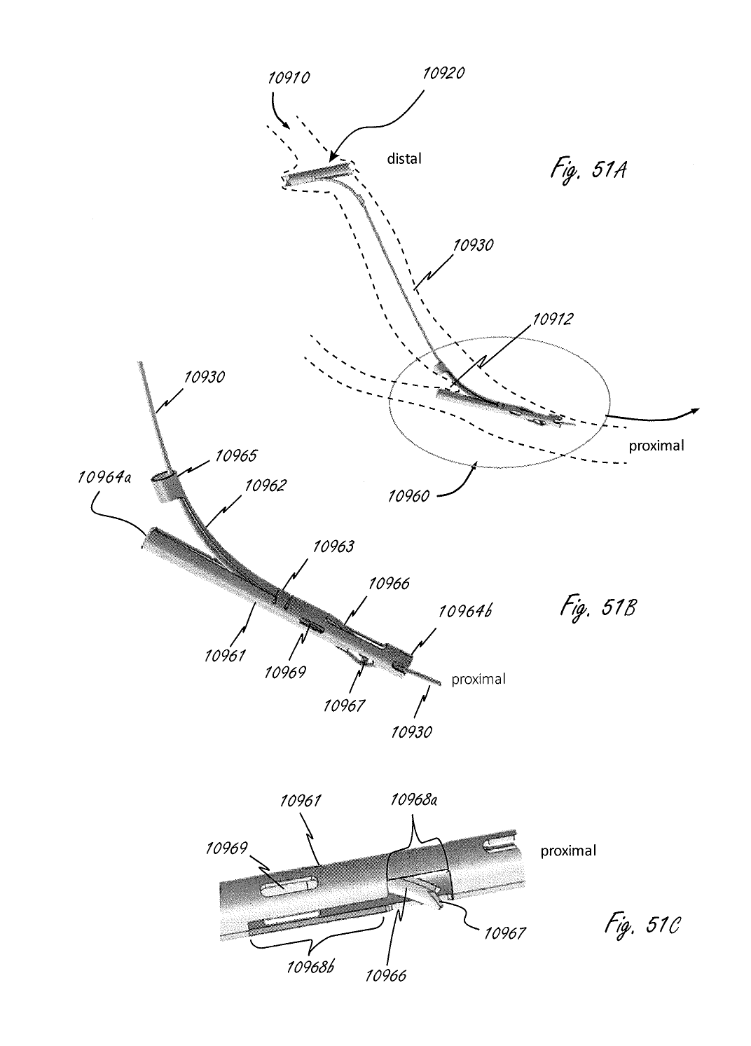

The tension tether line 10730 so far is conceived to be comprised of a relatively inelastic polymer or metal. As described above, the line of the same material could be made elastic by forming it into a coil or a braid, particularly with a process that imparted a permanent set, and/or added an elastic polymer matrix. The line material could alternatively be fabricated from an elastic rubber such as silicone or polyurethane. Preferably the line is itself radiopaque or in the case of a polymer, has a radiopaque additive known in the art. Coiling or braiding the line also helps increase the x-ray density of the radiopaque material for improved fluoroscopic visualization. Elasticity in the line material and/or the coiled or braided form of the line material would allow for some natural compliance in the airway to mimic the properties of the lung tissue. The line material could be set in a coil shape through a permanent mechanical strain in the wire, such as used to form a stainless steel coil, or via a high temperature heat setting process appropriate for nitinol (e.g., 504° C. for 5 min), or via heat setting a high strength polymer. A coil could also be fabricated by laser cutting or etching the coil pattern in a metal tube. As illustrated in FIG. 50, the distal anchor, tether line, and proximal anchor could all be laser cut or etched from a single metal tube (preferably nitinol).

For initial deployment, the operator advances the pusher tube or grasper to push the distal anchor, and preferably, the coiled tension tether line into the airway lumen. Once deployed, the pusher tube may be removed. At this point, the tensile line 10750 or grasper may be tensioned to transfer tension to the tether line and distal anchor. Preferably the delivery system is retracted the same amount as the proximal anchor within it. The bronchoscope may also require some degree of retraction. With fluoroscopic guidance, when the appropriate amount of tension and or movement of lung tissue with the distal anchor is achieved, the delivery system may be retracted relative to the proximal anchor such that the proximal anchor is deployed in the airway. The spring force of the tether extension 10742 and/or the off-center position of the attachment point 10743 helps the tubular structure 10741 rotate relative to the tether line 10730. Relaxation of the tension should allow the proximal anchor edge 10744a to impinge against and further rotate within the airway wall to secure itself. Alternatively, the user may wish to hook the distal end of the proximal anchor into an adjacent airway branch where it rotates and secures itself, using the carina as a lever point. Preferably the proximal anchor is deployed in a region where the bronchoscope can confirm the position in addition to fluoroscopy. Deflection of the bronchoscope in proximity to the proximal anchor may also aid in the relative rotation of the proximal anchor relative to the tension tether line. Adjustment of the proximal anchor position to a more proximal location may be accomplished by simply retensioning the proximal anchor and withdrawing it further before relaxing tension. Adjustment of the proximal anchor to a more distal position may require retracting the proximal anchor into the delivery system before relaxing tension, moving more distally, and then retracting the delivery system relative to the proximal anchor to deploy the proximal anchor in the more distal location. In some cases, the use of the delivery system may not be necessary depending on the angle of the airway. Once the desired proximal position is achieved, the tensile line 10750 may be severed or otherwise disengaged. If using a grasper, the grasper may simply be opened (while outside the delivery system and/or scope working channel) to release the proximal anchor and then closed and retracted through the scope. After the procedure, the physician may non-invasively monitor the tension and associated lung volume change between the anchors by fluoroscopically comparing the anchor position, orientation, separation distance, and/or slack level in the tether line, to the values immediately post procedure. Adjustments during follow-up procedures (to increase or decrease tension) could be achieved with graspers in the manner described above, preferably under direct bronchoscopic visualization, but also with fluoroscopic guidance. Complete reversibility of tension could also be achieved by severing the tensile tether line using conventional endoscopic tools. The proximal anchor could be removed with graspers. The distal anchor may possibly be removed by advancing the delivery catheter to the distal site and use small graspers or other tools to retrieve the distal anchor under fluoroscopic guidance.

An alternative method of deployment would be to use a guidewire as a support member passing through the distal and, if applicable, proximal tubular anchor as the anchor is advanced forward. For example, a 0.035″ guidewire could be used to pass through the 0.040″ inner diameter of the distal and/or proximal anchor tubular member. The guidewire could stay within the delivery catheter tube used to house the anchors as the anchors are advanced into the airway lumen, or the guidewire could be advanced beyond the delivery catheter tube to the location for anchor deployment. Alternatively, the anchors could reside solely on the guidewire without the use of a delivery catheter as the anchors and guidewire are advanced out of the working channel of the bronchoscope to the anchor deployment site. The pusher tube could then be passed over the guidewire to deploy the anchor(s).

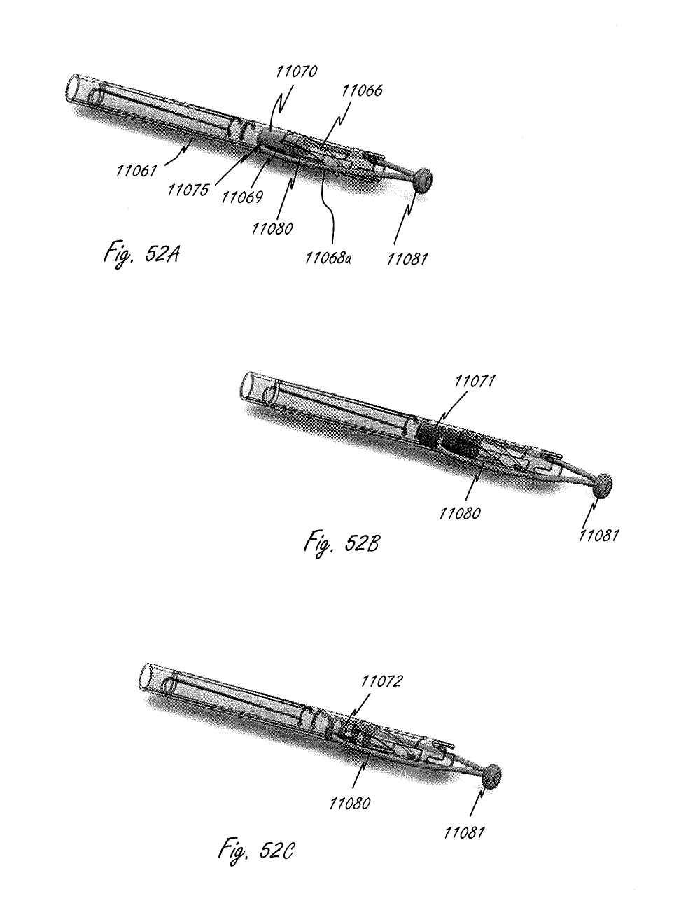

In the above embodiments, the tension tether line 10730 is fixedly attached to the proximal anchor. Another exemplary embodiment illustrated in FIGS. 51A-51C shows how the tether line may be ratcheted through an alternative proximal anchor. As illustrated in FIG. 51B, the proximal anchor 10960 is formed from a tubular element 10961. The distal end of the tubular element is formed such that a deflector arm 10962 having a distal ring 10965 deflects away from the distal tube 10964. The tether line 10930 passes through the distal ring 10965 attached to the deflector arm 10962 and continues inside the proximal end of the tubular element. The distal ring 10965 may be a solid tubular element as illustrated, or a formed ring of wire, or a coil to make the part more flexible during delivery. The deflector arm is preferably formed (via heat set or other process) such that it has a bias to move away from the longitudinal axis of the tubular element, and can have a curved configuration. This would help ensure the proximal anchor straddles the carina 10912 to aid it in anchoring. Anchoring is also (or alternatively) aided by the rotation of the tubular element 10961 within the airway 10910 while under tension, similar to the way the previous “T” anchors have been described, such that ends 10964a and/or 10964b press against the airway wall to constrain the movement of the proximal anchor. The tension tether line 10930 is releasable secured using ratchet pawl 10966 which is shaped to have a downward spring force against the line 10930. As illustrated in the underside of the part in FIG. 51C, a groove feature 10967 is provided in the end of pawl 10966 to increase the contact with the line and help constrain the line against the pawl. To achieve a meaningful deflection of the line, a lower window 10968a is formed in the tube through which the pawl and line may pass. The length and stiffness of the pawl as well as the length of the window opening 10968a both proximal and distal to the pawl are optimized to facilitate the level of engagement of the line. An optional groove 10968b slightly wider than the width of the line may be provided to allow the line to slack below the tube. This may be useful if the line needs to pass under a mechanism in the tube to deflect the pawl upwards, such as that described in FIG. 52A below. Because the pawl 10966 is angled down in the proximal direction, tensioning of the line from the proximal side allows the pawl to deflect upward and allow the line to slide past it. However, when the proximal tension is relaxed and instead applied from the distal end, the pawl deflects downward and the line bites into the pawl and tries to drag it down further and distal, further increasing the force on the line and preventing the line from sliding past. A lip at the distal end of the window 10968 may be provided to prevent the pawl from being deflected too far in the distal direction. The function of the pawl allows the proximal anchor to be pushed distally down the line while tensioning it from the proximal end, but once the proximal tension is relaxed, the tension from the pull of the distal anchor takes over and prevents further movement relative to the line.

FIGS. 52A-52C illustrate a mechanism for how the pawl 11066 may be disengaged from the line 11030 to allow it to be more easily advanced or retracted relative to the line. FIG. 52A illustrates a solid piston 11070 inside the tubular member 11061 with a transverse through hole 11075 aligned with the distal end of slot 11069 on the tubular member 11061. An engagement line 11080 is passed through the hole 11075 and slot 11069. As illustrated, the ends of the engagement line are secured within a bead 11081. The bead allows for releasable engagement by a grasping tool. In an alternative embodiment, the line 11080 could be instead be secured to itself or within another object with a different shape such as a cylinder, circular ring, “T” or “J” shaped hook. The bead could be attached using previously described processes, including those described for FIG. 47. Once grasped, the engagement line 11080 may be tensioned to slide the piston 11070 proximally to deflect the pawl upward to release the line 11030. The length of the slot 11069 controls the travel of the piston. FIG. 52B shows an alternative embodiment where a tubular piston 11071 is used instead of a solid piston. This allows the line 11030 to pass through the middle of the tubular piston 11071 such that it is more tightly constrained in the window 61108a. A slot in the tubular piston 11071 allows for engagement of the tube with the engagement line 11080. FIG. 52C shows another embodiment where a spring coil piston 11072 with an inner lumen is instead used to allow the line 11030 to pass through it. The spring may also provide more of a natural push back to its original position after tension is released. For the embodiments of 11010a or 11010b, the spring back may be accomplished by either pushing the line 11080 in a distal direction, or having the distal end of the piston attached to an elastic element (not shown) which stretches when the piston is pulled proximally and retracts the piston when tension is released.

The engagement line 11080 may be formed of any of the materials described for the tether tension line 11030 and would have a similar outer dimension. To also allow rigidity if compressed, it could be formed from a metal wire or strip, such as nitinol, stainless steel, or a ductal metal easily shaped. Biocompatible radiopaque materials such as metals (platinum, tungsten, tantalum, etc.) or polymers containing radiopaque additives (powders of barium sulfate, bismuth subcarbonate, tungsten, etc.) could be used for the engagement line 11080, piston (11070, 11071, 11072), and/or bead 11081.

An alternative method of disengaging the pawl is to compress the line and the pawl from the bottom such that the line is seated approximately within the space circumscribed by the outer diameter of the tubular element 11061. One means of accomplishing this would be by sliding a tube with a diameter just slightly larger than the outer dimension of tubular element 11061 over the pawl region. This could be done by pulling the proximal end of the anchor 11060 into the delivery device, or by sliding a separate tube slidably attached over the tubular element over the pawl region. In the latter example, the slidable outer tube could be attached to a bead and engagement line similar to that described in FIG. 52.

In a particular embodiment, the tubular element 11061 of proximal anchor 11060 is formed by laser cutting a nitinol tube. In one particular nonlimiting embodiment, the nitinol tube may be about 0.050″ OD with about a 0.005″ wall. The pawl may have a width of an outer arc length of 0.030″ and length of 0.120″. The window 11068a may have a circumferential arc width of 0.040″ and length of approximately 0.060″. The pawl position is set such that the pawl end 11067 passes through the plane of window 11068 formed by the outer diameter of tubular element 11061. The total length of the proximal anchor 11060 (such as when it is constrained within the delivery system) is approximately 0.5″. The deflector arm 11061 (including distal ring 62) is about 0.32″, with the distal ring being about 0.04″ long. The deflector arm width is approximately 0.03″ wide (on an outer circumferential arc), but could also taper proximal to distal from 0.040″ to 0.020″. As noted for other embodiments for the distal and proximal anchor, the materials and dimensions could be modified to optimize performance. A family of device sizes may also be provided to allow physicians to tailor placement to a particular anatomic location.

In another embodiment, a component comprising just the ratchet portion of the proximal anchor 11060 described above (such as just the portion illustrated in FIG. 51C) could be used to secure a separate component serving as either the proximal or distal anchor. For example, the expandable anchor described in FIG. 39 could be expanded by tensioning a tension tether line attached to the distal end of the anchor while compressing the ratchet portion described above against the proximal end of the expandable anchor. For an expandable anchor acting as a distal anchor, the ratchet securement would allow for reversing the expanded portion in order to remove the anchor if desired. A similar use could be performed to aid in controlling the amount of expansion of other expandable anchors such as the hinged T-anchors illustrated in FIGS. 35-36. A similar ratchet portion could be used on the distal and/or proximal side of an expandable proximal anchor. Also, as illustrated in FIG. 42E, multiple lines or pull wires from multiple distal anchors could pass through the ratchet feature (described previously as clamping bead 10011), with a single pawl engaging more than one line, or separate pawls provided for separate lines.

The implant embodiments above have so far been described as non-resorbable. However, there may be advantages to forming any or all of the distal anchor, proximal anchor, or tension tether line from a resorbable (also referred to as bioabsorbable) material. After the therapeutic effect of lung volume reduction and/or lung retensioning is achieved acutely, the lung tissue will remodel over time such that tension between the anchors is no longer needed to hold the tissue in the compressed state. In fact, the remodeling process may lead to a significant reduction or complete loss in tension in the line between the anchors. In this case, the anchors may dislodge and become an irritant. Also, if the anchors are not well encapsulated by the tissue, or bacteria is trapped in a biofilm against the implant, recurrent or persistent infection or pneumonia may develop. Another possible side effect of the prolonged presence of an implant is inflammation that becomes symptomatic for the patient. Inflammation and fibrosis around the implant may also lead to stiffening of the lung tissue, reducing its natural compliance and recoil, particularly if the inflammation and fibrosis progresses into otherwise healthy nearby lung tissue. Providing anchors and/or a tension line formed from a resorbable material, the implant will no longer be present in sufficient mass to pose the clinical issues described above. Examples of resorbable materials are described below, along with methods of dialing in the timeframe after which the appropriate strength and mass content may be allowed to decline.

Suitable biocompatible non-absorbable polymers for the distal anchor, proximal anchor and the connector (or tether line) include but are not limited to polyesters (such as polyethylene terephthalate and polybutylene terephthalate); polyolefins (such as polyethylene and polypropylene) polyisobutylene and ethylene-olefin copolymers); Teflon (PTFE,e-PTFE and their derivatives), polymethyl methacrylate, acrylic polymers and copolymers; vinyl polymers and copolymers; polyvinylidene halides, Polyvinylidene fluoride and polyvinylidene chloride; polyacrylonitrile; polyvinyl ketones; Polyether ether ketone (PEEK), polyaryletherketone (PAEK), liquid crystalline polymers, polysulfones, polyamides (such as nylon 4, nylon 6, nylon 66) polycarbonates; polyurethanes, silicones and siloxanes and fiber reinforced biocompatible non-absorbable polymers.

Suitable biocompatible bioabsorbable aliphatic polyesters for the distal anchor and proximal anchor include homopolymers and copolymers of lactic acid, glycolic acid, lactide, glycolide, para-dioxanone, trimethylene carbonate, ε-caprolactone or a mixture thereof. For the purpose of this invention aliphatic bioabsorbable polyesters include polymers and copolymers of lactide (which includes lactic acid d-, l- and meso lactide), ε-caprolactone, glycolide (including glycolic acid), hydroxybutyrate, hydroxyvalerate, para-dioxanone, trimethylene carbonate (and its alkyl derivatives), or a mixture thereof. The ratio of the homopolymers in the copolymer can be adjusted for mechanical performance, degradation profile and absorption characteristics. For example, while copolymers containing lactic acid and glycolic acid provide higher stiffness and strength, a higher proportion of lactic acid will allow for longer strength retention. Thus, while a 10/90 lactic acid/glycolic acid copolymer will retain about 75% strength at 2 weeks and about 25% at 2 weeks, 90/10 lactic acid/glycolic acid copolymer will retain about greater than 70% strength at 6 months and about greater than 40% between 9 months to 1 year. On the other hand, homopolymers of glycolic acid and copolymers rich in glycolic acid will resorb faster in the body compared to homopolymers of lactic acid and copolymers rich in lactic acid. Since it is not only important that the device materials offer adequate strength and stiffness at the time of implantation but also during the healing process with progressively lower load bearing demand, homopolymers and copolymers of para-dioxanone which can retain about greater than 40% strength at 6 weeks and resorbs in about 4 to 5 months can be a reasonable choice.

Further, biocompatible bioabsorbable polymers for the distal anchor and proximal anchor include aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, tyrosine-derived polyarylates or a mixture thereof. The biocompatible bioabsorbable homopolymers and copolymers used for the distal anchor and proximal anchor can undergo hydrolytic degradation under physiological conditions in the body, and will get resorbed in a biocompatible manner over time. The resorption of these polymers follows very well-known pathways. In one embodiment, the biocompatible bioabsorbable polymers can be reinforced with short or long fibers for increasing their load bearing capabilities.

The distal anchor and proximal anchor can be made by a variety of process including but not limited to extrusion, coextruding, injection molding, insert injection molding, compression molding and short and long fiber composite making processes. The distal anchor and proximal anchor can be made by machining of desired anchor geometry from polymer stock or blocks. Laser cutting patterns or shapes in the material may also be performed, which is particularly applicable for creating expandable stent-like anchor structures from extruded tubes.

In another embodiment, the distal anchor and proximal anchor made from bioabsorbable homopolymers and copolymers, maintains at least 90% of its load bearing capacity in vivo for at least 2 weeks. In another embodiment, distal anchor and proximal anchor made from bioabsorbable homopolymers and copolymers, maintains at least 50% of its load bearing capacity in vivo for at least 2 weeks. In another embodiment, distal anchor and proximal anchor made from bioabsorbable homopolymers and copolymers, maintains at least 40% of its load bearing capacity in vivo for at least 6 weeks. In another embodiment, distal anchor and proximal anchor made from bioabsorbable homopolymers and copolymers, maintains at least 90% of its load bearing capacity in vivo for at least 3 months. In another embodiment, distal anchor and proximal anchor made from bioabsorbable homopolymers and copolymers, maintains at least 50% of its load bearing capacity in vivo for at least 3 months. In another embodiment, distal anchor and proximal anchor made from bioabsorbable homopolymers and copolymers, maintains at least 60% of its load bearing capacity in vivo for at least 6 months. In another embodiment, distal anchor and proximal anchor made from bioabsorbable homopolymers and copolymers, maintains at least 50% of its load bearing capacity in vivo for at least 12 months.

In another embodiment, distal anchor and proximal anchor made from bioabsorbable homopolymers and copolymers, are substantially resorbed in a biocompatible manner within at least 3 months. In another embodiment, distal anchor and proximal anchor made from bioabsorbable homopolymers and copolymers, are substantially resorbed in a biocompatible manner within at least 12 months. In another embodiment, distal anchor and proximal anchor made from bioabsorbable homopolymers and copolymers, are substantially resorbed in a biocompatible manner within at least 24 months.

Suitable biocompatible bioabsorbable aliphatic polyesters for connector (or tether line) include homopolymers and copolymers of lactic acid, glycolic acid, lactide, glycolide, para-dioxanone, trimethylene carbonate, ε-caprolactone or a mixture thereof. For the purpose of this invention aliphatic bioabsorbable polyesters include polymers and copolymers of lactide (which includes lactic acid d-, l- and meso lactide), ε-caprolactone, glycolide (including glycolic acid), hydroxybutyrate, hydroxyvalerate, para-dioxanone, trimethylene carbonate (and its alkyl derivatives), or a mixture thereof. The ratio of the homopolymers in the copolymer can be adjusted for mechanical performance, degradation profile and absorption characteristics. While the connector (or tether line) will require adequate stiffness and strength to maintain load between the anchors and the proximated tissues, it also needs flexibility thus possibly preferring polymers and copolymers rich in caprolactone and para-dioxanone, materials with elongation to break greater than 25% or even 50%. Thus, homopolymers and copolymers of para-dioxanone which can retain about greater than 40% strength at 6 weeks and resorbs in about 4 to 5 months can be a reasonable choice. A copolymer of about 60 to 80% caprolactone and about 20 to 40% lactic acid while providing retention of about greater than 90% of its load bearing capabilities for about 3 to 4 months, about greater than 75% of its load bearing capabilities for about 6 to 9 months also can be a candidate for the connecting string. A copolymer of about 15 to 30% caprolactone and about 65 to 85% glycolic acid while providing retention of about greater than 60% of its load bearing capabilities for about 2 weeks, about greater than 25% of its load bearing capabilities for about 3 weeks also can be a candidate for the connecting string. It is likely that the polymers and copolymers rich in caprolactone and para-dioxanone are in the form of single filament or filament type geometry. The connecting string can also me made from stiffer polymers such as homopolymers and copolymers containing higher concentrations of lactic acid and glycolic acid but then the connector will be need to me made from muti-filament braids.

Further, biocompatible bioabsorbable polymers for connector (or tether line) include include aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, tyrosine-derived polyarylates or a mixture thereof. The biocompatible bioabsorbable homopolymers and copolymers used for the distal anchor and proximal anchor can undergo hydrolytic degradation under physiological conditions in the body and will get resorbed in a biocompatible manner over time.

In another embodiment, the connector (e.g., tether line) can be made by a variety of process including but not limited to extrusion, coextruding, injection molding or other fiber making processes. Fiber making processes can include textile operations such as braiding, knitting and weaving. The connector (or tether line) can be a mono-filament, multi-filament, co-mingled filaments or yarns. In one embodiment, the connector (or tether line) can be flexible. In one embodiment, the connector (or tether line) is load bearing. In one embodiment, the connector (or tether line) is elastically resilient. In one embodiment, the connector (or tether line) is flexible. In one embodiment, the connector (or tether line) is not elastomeric. In another embodiment, the connector (or tether line) can form a loop, a knot, a partial loop, an adjustable loop, a movable knot, a pushable knot.

In one embodiment, the connector (e.g., tether line) is made from bioabsorbable polymers and copolymers, maintains at least 90% of its load bearing capacity in vivo for at least 2 weeks. In another embodiment, the connector (or tether line) made from bioabsorbable polymers and copolymers, maintains at least 50% of its load bearing capacity in vivo for at least 2 weeks. In another embodiment, the connector (or tether line) made from bioabsorbable polymers and copolymers, maintains at least 40% of its load bearing capacity in vivo for at least 6 weeks. In another embodiment, the connector (or tether line) made from bioabsorbable polymers and copolymers, maintains at least 50% of its load bearing capacity in vivo for at least 3 months. In another embodiment, the connector (or tether line) made from bioabsorbable polymers and copolymers, maintains at least 40% of its load bearing capacity in vivo for at least 6 months. In another embodiment, the connector (or tether line) made from bioabsorbable polymers and copolymers, maintains at least 30% of its load bearing capacity in vivo for at least 12 months.

In another embodiment, the connector (e.g., tether line) made from bioabsorbable polymers and copolymers, are substantially resorbed in a biocompatible manner within at least 3 months. In another embodiment, the connector (or tether line) made from bioabsorbable polymers and copolymers, are substantially resorbed in a biocompatible manner within at least 12 months. In another embodiment, the connector (or tether line) made from bioabsorbable polymers and copolymers, are substantially resorbed in a biocompatible manner within at least 24 months.

In some embodiments, the distal anchor and the proximal anchor can be all made from bioabsorbable polymers and the connector (e.g., tether line) can be all made from biostable polymers. In another embodiment, the distal anchor and the proximal anchor can be all made from biostable polymers and the connector (e.g., tether line) can be all made from bioabsorbable polymers.

In other embodiments, it may be desirable or necessary to use a non-resorbable metal or polymer in conjunction with a resorbable material. For example, the strength of a barb or pawl may require a non-resorbable component to hold in the tissue or against the tether line, but the surrounding structure of that feature may be resorbable so that a minimal amount of non-resorbable material is left behind that can be encapsulated in the tissue or easily retrieved without significant clinical sequelae. In another example, a non-resorbable radiopaque material or compound (e.g., barium sulfate, bismuth subcarbonate, tungsten, tantalum, platinum, gold) may be mixed into at least a portion of the resorbable polymer matrix for fluoroscopic visualization of the part as well as fluoroscopic monitoring of changes in the shape of the part that could indicate the degree of resorption. A separate radiopaque component may also be attached, insert molded, coextruded, press-fit, or otherwise integrated into the resorbable material.

The exemplary embodiments below describe methods and devices that couple at least one outer implant (also referred to herein as an outer implant member) to at least one inner implant.

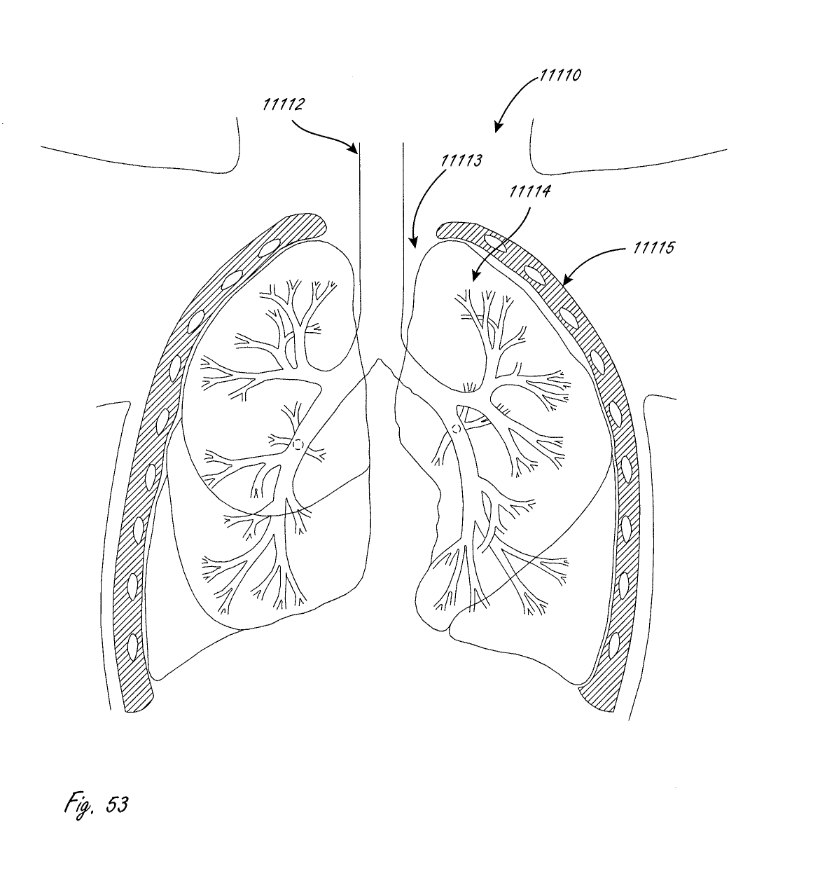

FIG. 53 illustrates a pulmonary anatomy 11110 comprising lung bronchus (or airway) 11112 and surrounding lung tissue 11113 within chest wall 11115.

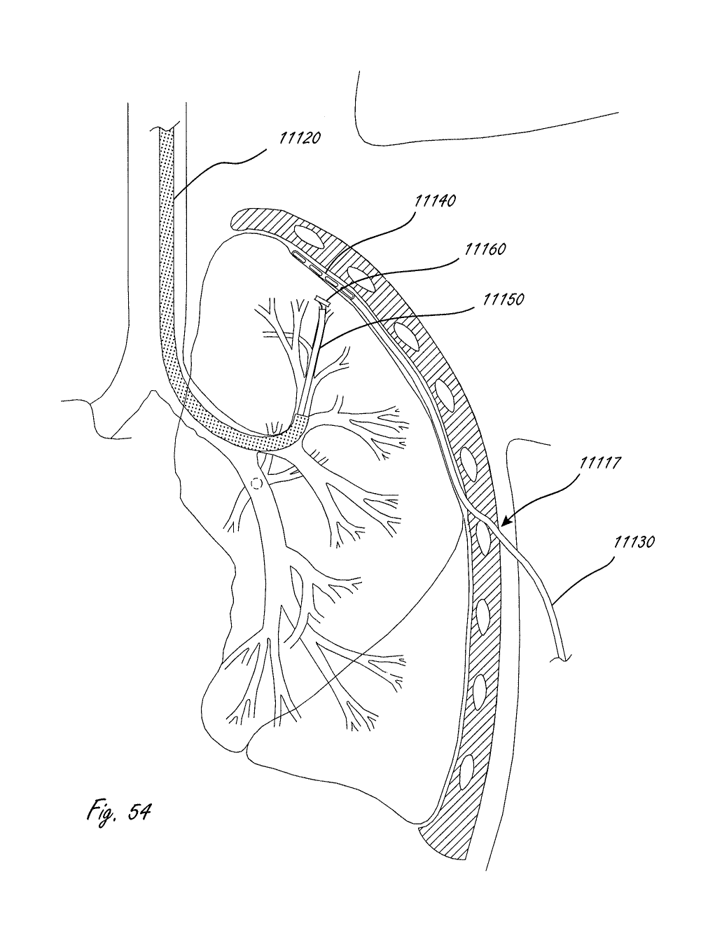

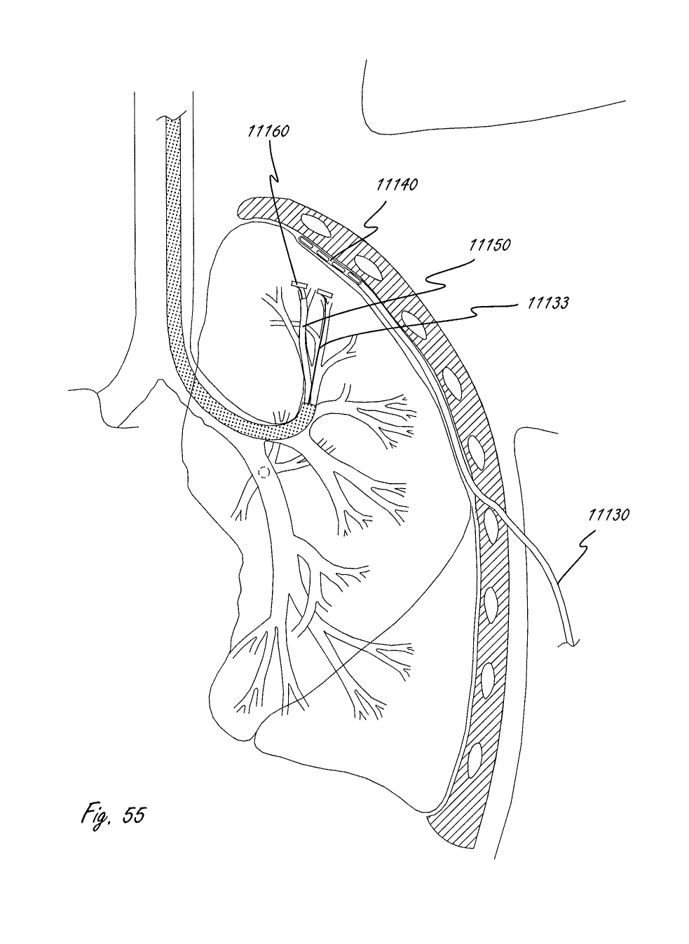

As illustrated in FIG. 54, a pleural delivery catheter (PDC) 11130 containing outer implant 11140 is introduced into the pleural space. The method of introduction may be via thoracostomy, preferably at the “safe triangle”, using blunt dissection and tunneling over the top of the rib (such as is commonly performed for chest tube placement). Alternatively, access may be made via ultrasound guided initial needle insertion with subsequent guidewire advancement and sizing up to an introducer sheath (not shown). The introducer sheath, if used, preferably has an air-tight valve to minimize the chance of air introduction into the pleural space. A full surgical thoracotomy could also be used to facilitate outer implant placement. The PDC 11130 preferably has fluoroscopic markers and/or materials that allow the user to visualize the device on fluoro. The PDC may be advanced to the target outer lung location using any combination of fluoroscopic, ultrasound, or CT imaging. Electromagnetic navigation may also be employed using sensors in the PDC and any bronchoscopically delivered devices.

FIG. 54 also illustrates the delivery of the inner implant (also referred to as the distal anchor) 11160 using a bronchus delivery catheter (BDC) 11150 advanced from the working channel of a bronchoscope 11120. Fluoroscopic visualization of the PDC 11130 provides the user with a reference to direct the distal anchor placement and to avoid getting too close to the pleural edge of the lung where the risk of pneumothorax increases. The distal anchor 11160 may be configured in a variety of shapes and configurations previously disclosed to improve the ability of the anchor to hold tension. However, in certain embodiments of use, the anchor does not necessarily have to hold significant tension if all it is required to do is couple to the outer implant to compress the tissue and/or airspace in between the distal anchor and outer implant.

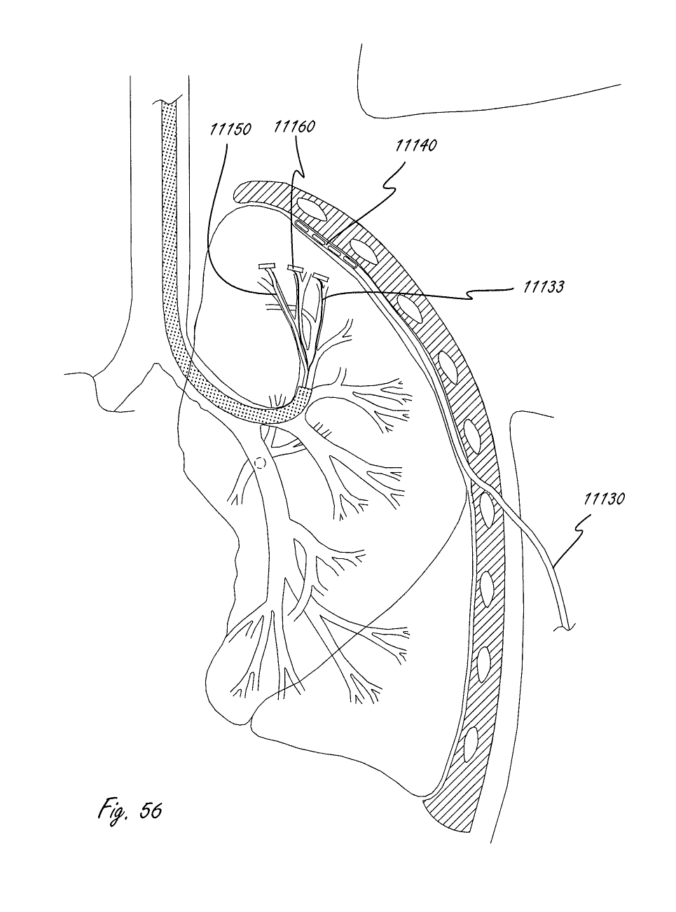

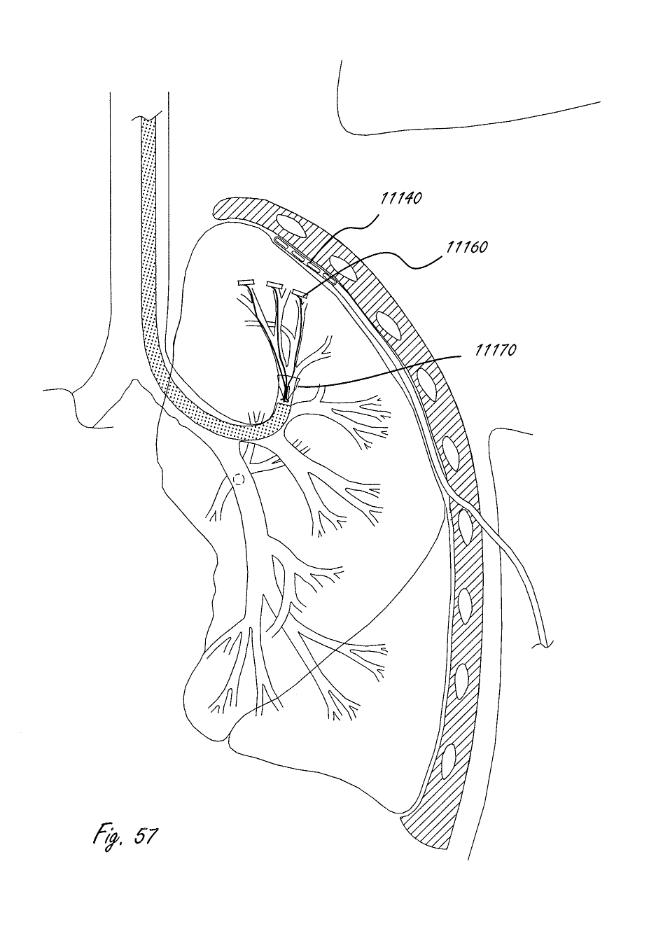

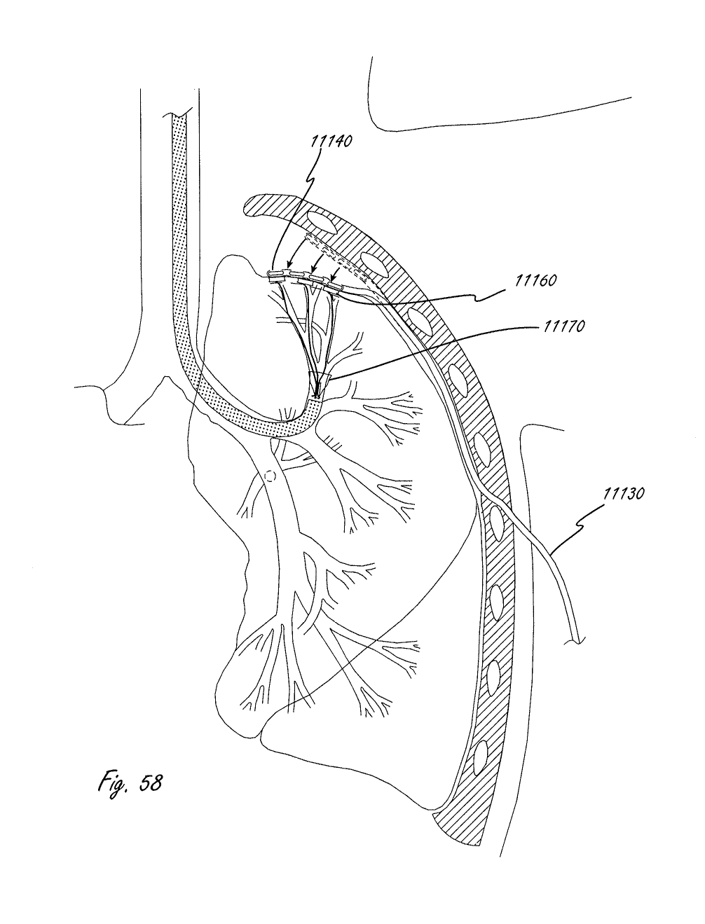

FIGS. 55 and 56 illustrate how more than one distal anchor 11160 (only one labeled) may be deployed within the bronchi. In addition to coupling to the outer implant 11140, these distal anchors may also be configured to couple to one another thus compressing volume between the corresponding bronchi. The BDC 11150 may incorporate a deflection mechanism to aid in moving the distal anchors close enough to one another to aid in coupling. In preferred embodiments, the distal anchors 11160 may include a tension tether line 11133 which extends to a proximal location accessible with a bronchoscope. The tether line 11133 may be useful to aid in delivery catheter advancement to re-adjust or remove the distal anchor. It also allows a means to further tension the coupled elements and attach to a proximal anchor 11170, such as that illustrated in FIG. 57-58. By holding the tether line in tension, the proximal anchor serves to further compress the space between the distal and proximal anchors. Preferably, the proximal anchor includes a ratchet feature to allow adjustments to the tension.

To enable coupling, the outer implant 11140 and distal anchor 11160 comprise one or more magnetic coupling elements (MCE). To achieve coupling, any given coupled pair of MCE may comprise a magnet and another magnet or a magnet and a magnetically attracted ferromagnetic element or alloy. The magnet portion is preferably a permanent rare earth magnet such as neodymium (also known as NIB or Nd2Fe14B), or samarium-cobalt (SmCo5). Where two magnets are used, the opposite poles are oriented toward one another for attraction. A given MCE may be comprised of a single element or a stack or group of smaller elements. The smaller elements may have a given dimension (length, width, thickness, or diameter) in the range of 1-10 mm. In other embodiments, the smaller elements may have a given dimension of 0.1-1 mm or in some cases much smaller, such that they comprise a group of shavings, grains, or powder. The much smaller elements may also be provided suspended in a polymer matrix. In another embodiment, the much smaller elements may be provided in a paste-like substance, or adhesive such as cyanoacrylate, which adheres to the tissue as it is painted on. To improve integrity and biocompatibility, the MCE may be plated with an inert metal or coated with, or encapsulated within, a biocompatible polymer such as those comprising silicone or urethane and the like. The MCE may additionally be hermetically sealed in an inert metal housing, such as one formed of titanium, using a welding process.

In another embodiment, where remote control over the magnetic properties is desirable, an electromagnet could be used where a power source is supplied or disabled via induction and/or transmission through a transcutaneous lead. Power via transthoracic ultrasound transmission to a piezoelectric receiver is also possible.

As illustrated in FIG. 58, once both the inner and outer implants are positioned, the MCEs in the outer implant 11140 may be directed toward the MCE in the inner implant distal anchors 11160 with the aid of the PDC 11130. In one embodiment, the outer implant 11140 is mechanically deflected toward the inner implant using pull wires attached to a proximal user activated handle of the PDC. The deflection mechanism may be slidable within the PDC and outer implant, such that it may be removed from the outer implant after coupling is completed. In another embodiment, the PDC may be used to deploy a temporary expandable member that fills the pleural space and presses the outer implant into the inner implant. The expandable member may be a compliant or non-compliant balloon, a bladder, an expandable cage, an expandable coil, or other expandable members known in the art. After the outer and inner MCE are coupled, the expandable member may be collapsed and removed through or with the PDC. In another embodiment, there may be a clinical advantage of detaching the expandable element and leaving it within the pleural cavity in its expanded state to maintain compression of the hyperinflated lung tissue. It could also be re-engaged with a PDC, collapsed, and removed at a follow-up procedure.

Another way of coupling the outer and inner implant would be to purposefully collapse the lung by equalizing pressure between the pleural space and atmosphere via the PDC. Vacuum to the pleural space could be reapplied through the PDC to re-inflate the lung. Alternatively, a balloon catheter could be advanced into the desired bronchus, inflated to occlude the airway, and vacuum applied to the distal airway exceeding the vacuum in the pleural cavity. Deflation of the balloon re-establishes the pressure differential to re-inflate the lung. The collapse and re-inflation of the lung could be performed quickly to minimize any clinical sequela.

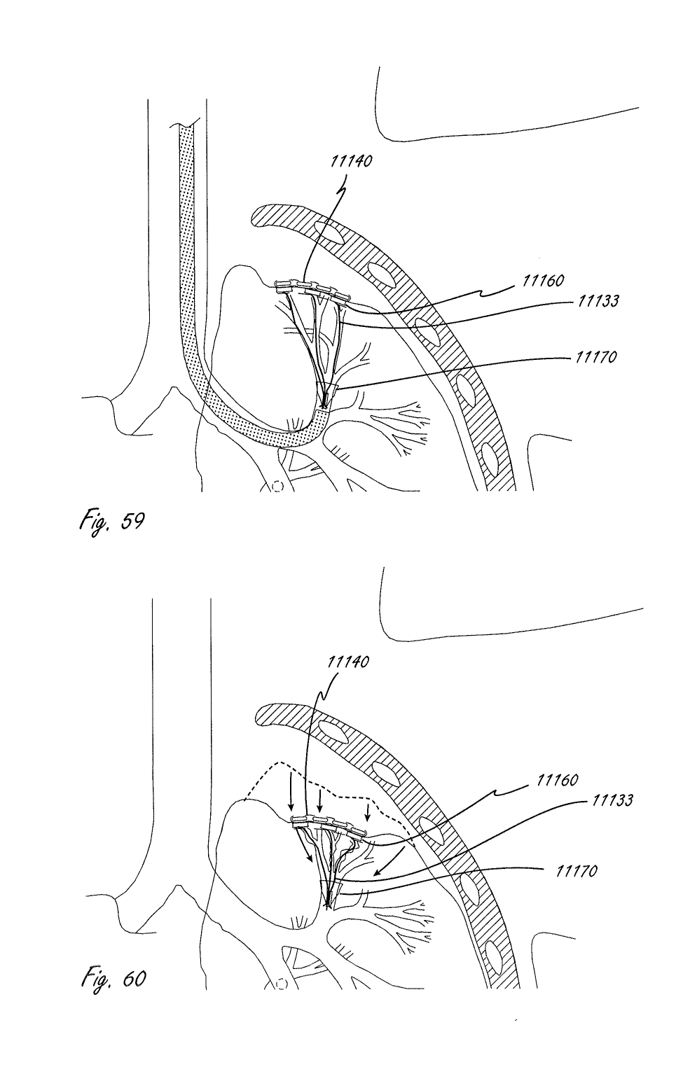

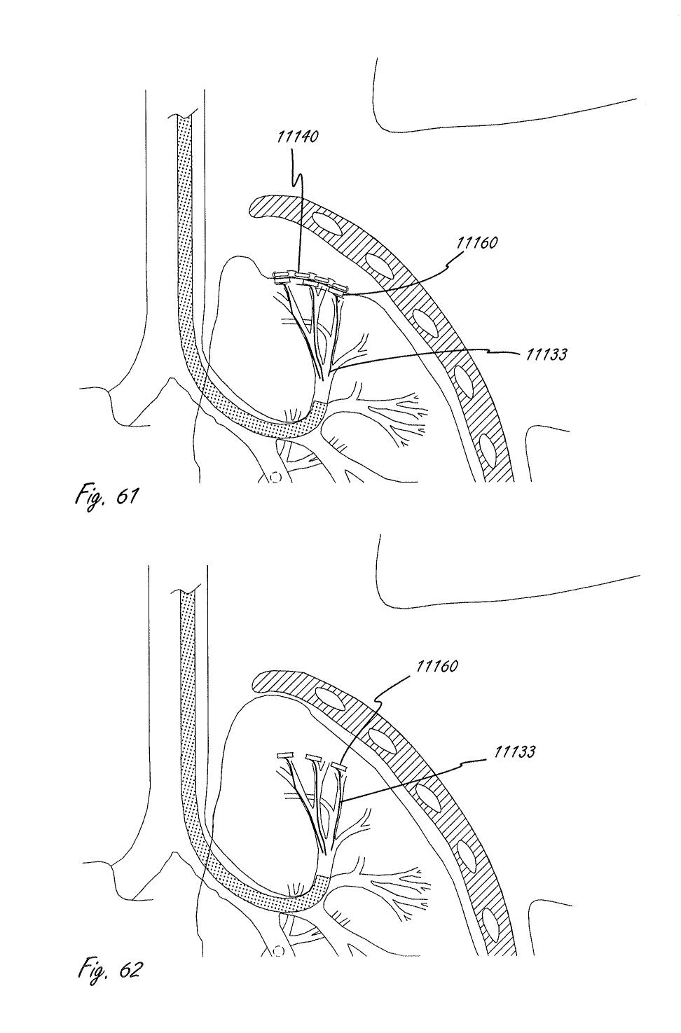

As shown in FIG. 59, once the inner and outer implants are coupled, the outer implant may be detached from the PDC 11130. This could be achieved through the removal of a line and slip knot holding the implant to the delivery catheter. A mechanical clip or grasper could also be released using a mechanism in the proximal handle of the PDC. FIG. 60 illustrates the coupled distal anchor 11160 and outer implant 11140 being tensioned together with the tension tether line(s) 11133 toward the proximal implant 11170 where the tension tether line(s) 11133 may be secured to the proximal implant.

While the proximal anchor 11170 may be placed once any or all of the distal anchors are placed, it may instead be placed after coupling of the outer implant and distal anchors. A separate proximal anchor may also be paired with each individual distal anchor. In another embodiment, the proximal anchor may also contain an MCE to aid in coupling the proximal anchor to the distal anchor (whether the distal anchor is coupled to the outer implant or not). As noted above, collapsing the lung may also aid in coupling the distal anchor and proximal anchor.

While delivery of the outer implant may occur first, followed by bronchoscopic delivery of the inner distal anchors, the order may be also reversed, or occur simultaneously. While delivery of both is preferably within a same-day procedure, it may be advantageous to deliver either the outer or inner implants on separate days, with up to a few weeks or months in between to allow time for the body tissue to remodel around the implants before purposely coupling the two implants. This remodeling may help prevent the implants from tearing loose prematurely and reduce the risk of pneumothorax.

FIG. 61 illustrates how the tension tether lines 11133 may be cut to release tension between the distal and proximal anchors. FIG. 62 illustrates how the outer implant may be removed with a PDC (not shown) in the event a patient has a reaction to the implant or exacerbation of emphysema.

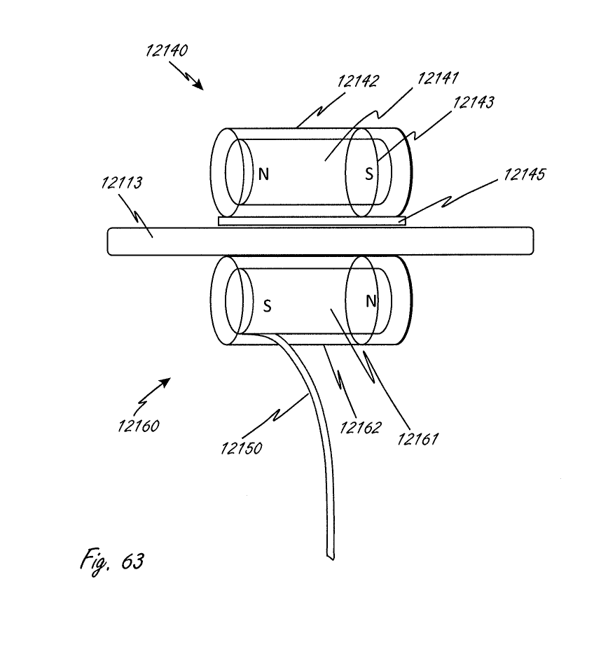

FIG. 63 illustrates an embodiment of the coupled outer implant 12140 and distal anchor 12160. In this case the outer MCE 12141 is encased in a housing 12142, such as a titanium shell, with a cap welded at location 12143. The housing 12142 could also be formed of silicone or other biocompatible polymer insert molded or dip/spray coated over the outside of the MCE 12141. A compressible polymer 12145 may be coupled to the housing to modulate the magnetic coupling force and distribute the load on the fragile outer lung tissue. The distal anchor is similarly configured with an MCE 12161 surrounded by a housing 12162 similar to that of the outer implant. A tether line 12150 attached to the distal anchor 12160 allows it to be tensioned and/or reaccessed/retrieved. In this embodiment, the MCE are cylindrical with the north/south axis of each approximately parallel to the coupled tissue 12113. Other shapes could also be employed. The center or the MCE could also be hollow to facilitate connection to delivery elements.

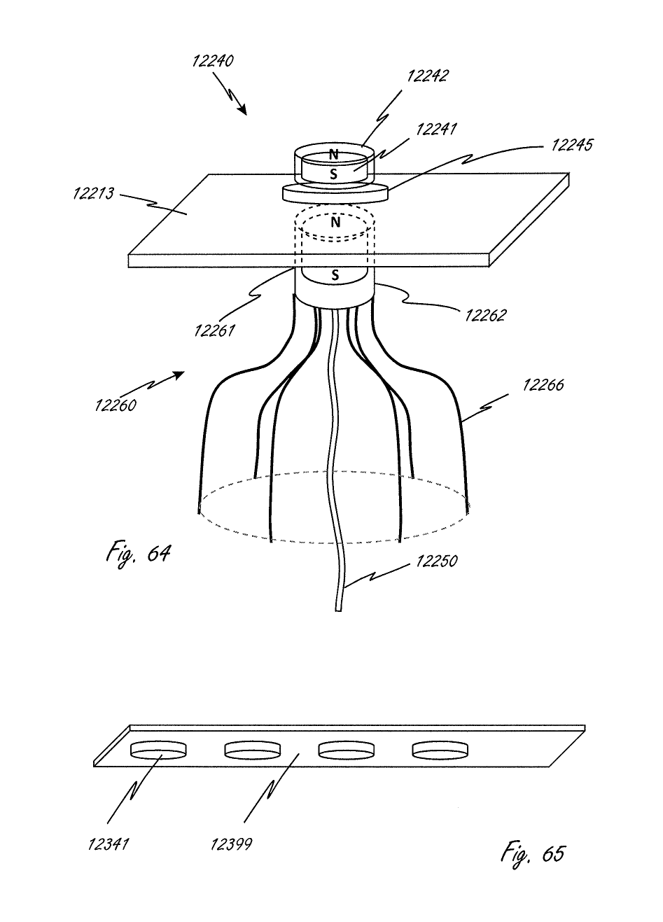

FIG. 64 uses similar materials to that described above, however the MCE have north/south axes approximately perpendicular to the coupled tissue 12213. Instead of the embodiment of FIG. 63, this distal anchor embodiment is shown with self-expanding anchoring elements 12266 to aid in anchoring. The arms could also be linked together in a stent-like pattern. The anchoring elements may be oversized for the lumen, and/or include tines, to further increase their hold on the tissue. A tether line 12250 attached at the distal region containing the MCE extends proximally from the center of the element.

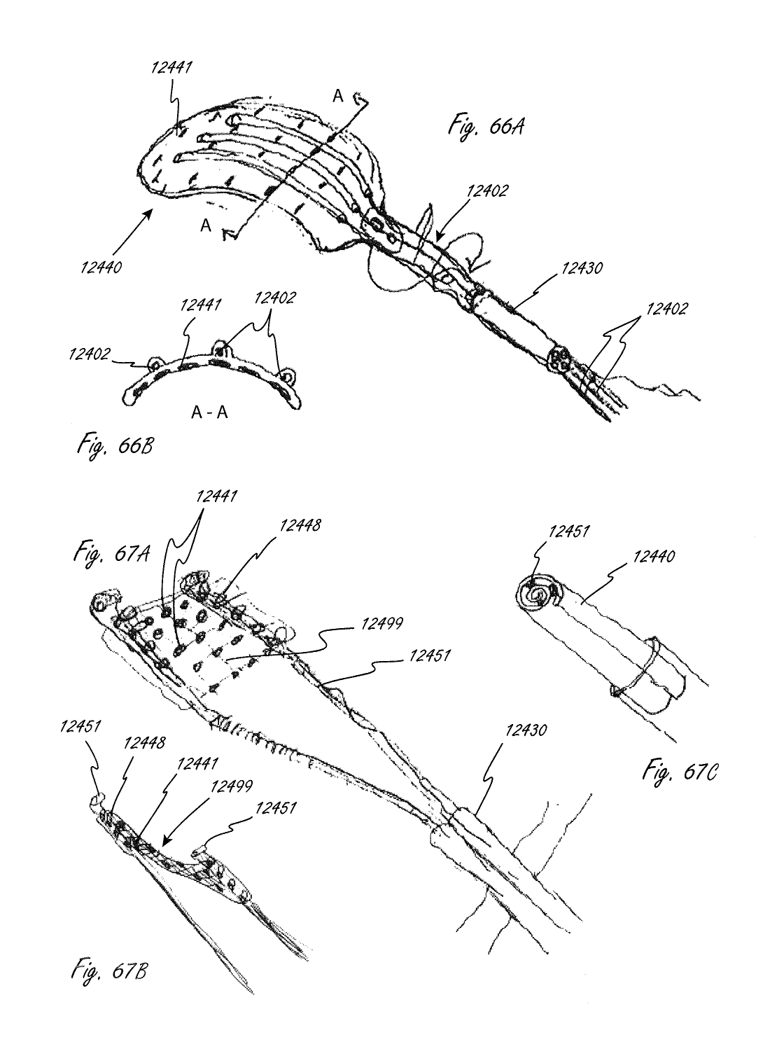

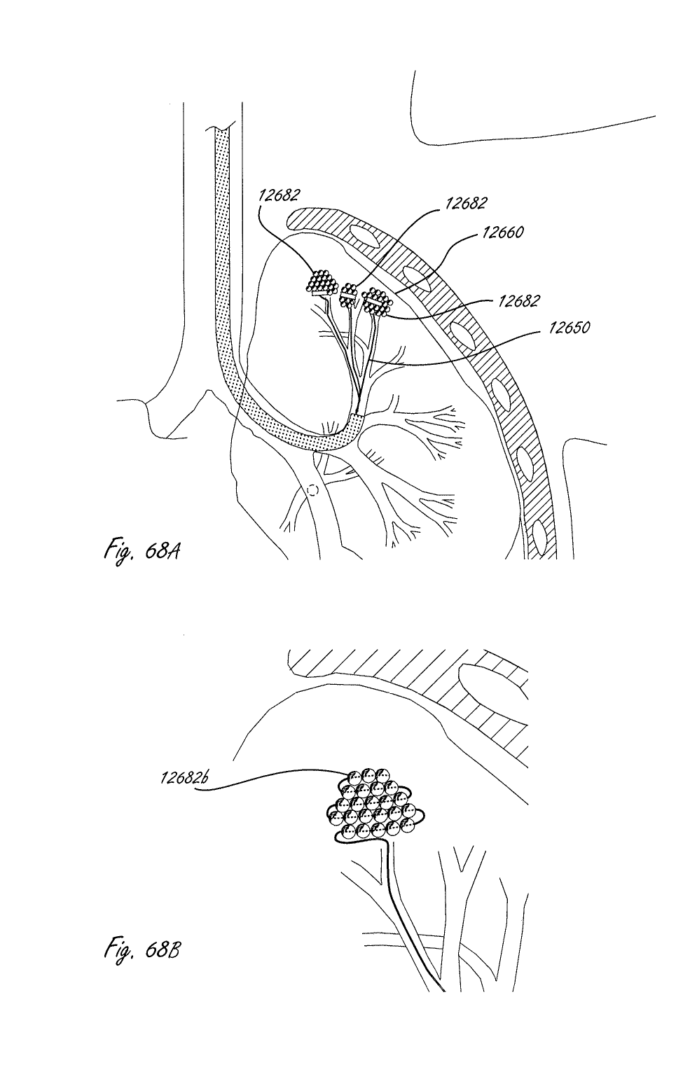

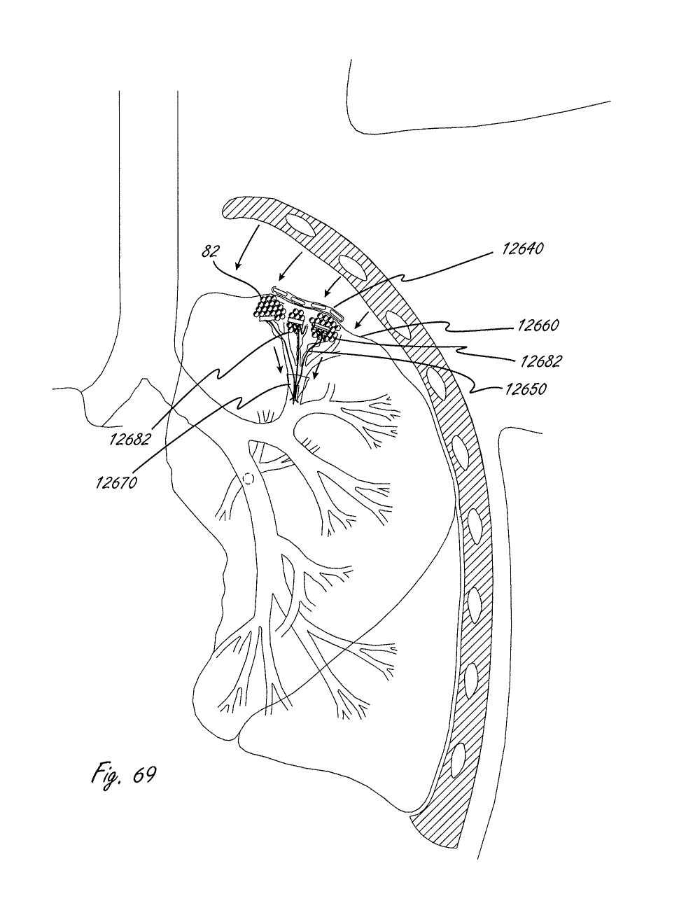

FIG. 65 illustrates how the outer implant may be a linear array of MCE 12341 constructed upon a molded matrix or netlike structure 12399 deployed sequentially over the tissue to couple with the inner implant.

FIGS. 66A-B and 67A-C illustrate two embodiments of how the outer implant 12440 may be constructed with multiple MCE 12441 in a 2-D array on a net-like structure or fenestrated elastic film 12499. Push arms 12451 are used to advance the net 12499 forward over the target tissue, and then may be released with a slip not line 12448 and retracted. The array could also be deployed in a rolled configuration, as shown in FIG. 68C, or staggered as shown in FIG. 68B to facilitate advancement through a smaller opening. 12430 illustrate at least a portion of a delivery device, such as at least one elongate shaft.

Another way of disposing the internal MCE would be to initially deploy a first MCE, followed by the deployment of additional MCE at the same site (e.g., via the same airway route). The first MCE could be anchored and have a tether line extending proximally as described above. In another embodiment, the first MCE would not necessarily be anchored in place, but rather just “dropped” at the site from a delivery catheter and not tensioned right away (regardless if a tether line is attached to the MCE or not). Successive MCE could then be advanced from the same delivery catheter and allowed to couple directly to the first MCE. In this way, a large MCE could be constructed from multiple MCE. This larger MCE would have the advantage of being able to anchor in the tissue due to it being oversized relative to the airway from which the MCE were delivered (the MCE could fill into an emphysemous cavity), and may also provide greater coupling than any individual MCE. The subsequent MCE delivered after the first MCE may or may not have tether elements or a grasping feature.

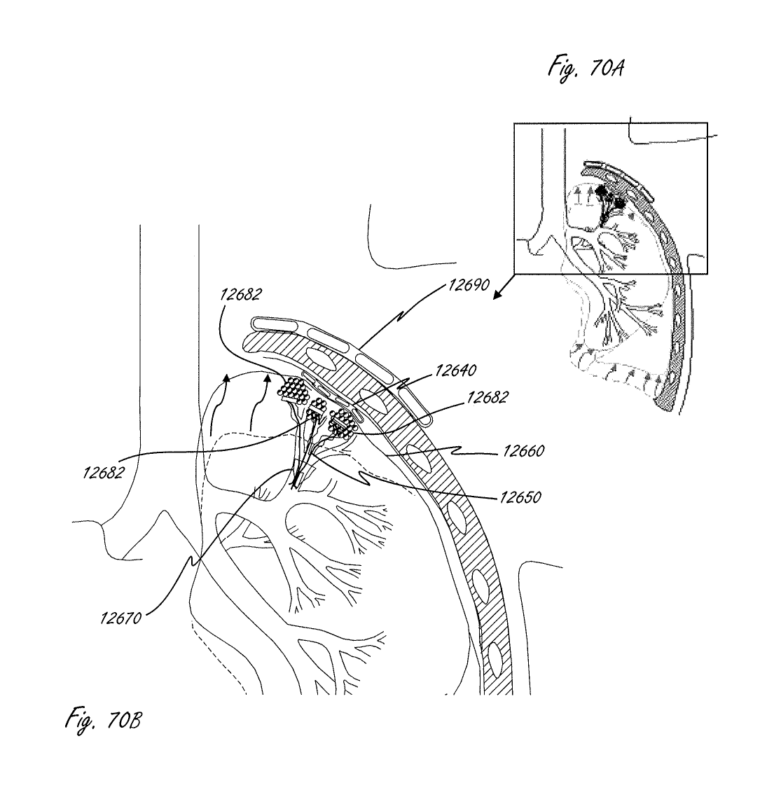

In an embodiment as represented in FIGS. 68A-70B, each MCE is a sphere 12682 of approximately 2 mm in diameter having a north and south magnetic axis (similar to the earth). A first sphere is pushed into the lung from the delivery catheter, followed by subsequent spheres. As illustrated in FIGS. 68A and 68B, the tendency will be for the spheres to bunch into a ball-like shape to create a larger MCE. This may be sufficient for attraction to other internal MCE or MCE within the pleural space or even outside the chest as will be described later. Preferably one or more of the spheres 12682 has a tether line 12650 for tensioning to a proximal anchor, as shown in FIG. 68B. The tether line 12650 also facilitates re-accessing the site to remove the spherical MCEs 12682 if necessary. One means of attaching the tether 12650 to the MCE would be to enclose the MCE in a hermetically sealed housing (as described previously) and bonding or welding the tether line to an opening in the housing into which the tether line extends. One or more of the MCE could also be captured within a flexible polymer tube or a braided tubular net cinched and secured around the MCE. FIG. 69 is similar to FIGS. 70A and 70B except the spherical MCE 12682 are shown coupled to and surrounding a tethered tubular MCE distal anchor 12660. The coupled groups of internal implant MCE are shown coupled to the pleurally positioned outer implant 12640.

In the above embodiment, MCE shapes other than spheres could also be employed. Examples include solid cylindrical or tubular elements which may or may not incorporate tines in a housing to facilitate anchoring in the tissue. The MCE may be deployed to bunch up in the lung or may be carefully deployed in a sequential manner to create a string of MCE in the lung. The tubular MCE, or any shaped MCE with a through lumen, could be deployed over a guidewire to control their position. Similarly, the MCE comprising a luminal element could be deployed over a line (such as a metal wire, polymer monofilament, or braid or coil of any combination of these materials) that remains with the MCE after deployment. The MCE may be free to move along the line, or be constrained individually or in groups with a knot or other similar physical constraint in the line. This would allow easier removal by pulling the proximal end of the line into a catheter lumen to extract the MCE from the lung. A similar construction could be used for MCE deployed in the pleural space. A combination of MCE shapes could also be used. For example, as illustrated in FIG. 68, an initial tubular MCE could be deployed in the target location and then spherical elements deployed around the tube. One advantage of the multiple MCE would be to provide a structure that could drain mucous or allow medication to reach sites distal to the implant site. An array of parallel tubular MCE or a tubular MCE surrounded by other MCE shapes would further enhance this ability. The magnetized axis (e.g., spanning the far ends of a tube vs. the sides of the tube) could be designed to properly orient the MCE in the desired shape.

Delivery of the internal implant may be made easier if the MCE elements are not magnetic at the time of delivery. This would prevent unintended coupling until the MCE are properly disposed. The MCE could then be magnetized after delivery by exposing them to a large external magnetic field to orient the dipoles. This may further serve to increase the strength of a group of internal MCE by creating a uniform orientation of the dipoles.

In another embodiment, in addition to or instead of, the outer implant 12640 disposed in the pleural space, the outer implant described above could be disposed on the outside of the chest wall, above the ribs. This outer implant (herein after referred to as the external MCE 12690) could be positioned either before, after, or during internal implant placement. The external MCE 12690 could also be positioned either before, after, or during a pleural implant 12640, if used.

A particular benefit of the external MCE 12690 would be to attract the internally implanted MCE 12660 within the lung toward the chest wall where they couple to the chest wall. This would serve to compress outer blebs and bullae between the inner and outer/external implants. The movement of the lung toward the chest wall may improve lung mechanics. This is particularly true s illustrated in FIGS. 70A and 70B, where the outer/external MCE position is optimized relative to the upper lobes such that the lung is drawn upward to reduce downward pressure on the diaphragm. The diaphragm optimal shape is restored for more efficient inspiration and expiration.

During a procedure, or within the first days and weeks following placement of the internal implant, it may be possible to manipulate the position of the external MCE to optimize lung compression for maximum patient lung mechanics and symptom relief. Where the MCE is external to the skin, the MCE may simply be moved in increments around the skin. MCE may also be added to or removed from the initial MCE to adjust the coupling strength (and thus degree of lung compression on the inside of the chest cavity). Where a pleurally placed MCE 12640 is already coupled to the internal MCE 12660, manipulation of the external MCE 12690 may aid in shifting the internally coupled elements to optimize lung compression. The external MCE 12690 may also aid in drawing the internal MCE 12660 to one another and/or to a pleurally placed MCE 12640. In one method of use, the external MCE 12690 could be removed once the desired internal and/or pleural MCE coupling is accomplished.

Placement of the external MCE 12690 could be accomplished by inserting the outer implant subcutaneously (or submuscularly) in the chest in proximity to the inner implant(s) to which it would couple. Another placement means would be to secure it to the epidermis in a similar region to provide a similar effect. Qne means of holding the external MCE in place would be with a skin adhesive known in the art, above which the MCE are disposed. Alternatively the MCE could be incorporated into a wearable fabric that could be held in place with a Velcro strap, snaps, or other well-known methods, or simply be worn like a vest. The fabric could also incorporate pockets/pouches disposed over its surface to allow the MCE to be swapped for stronger or weaker MCE as the patient need requires; alternatively the existing external MCE could be swapped for a different wearable set of MCE. Additional MCE could also be added (via magnetic coupling) to the existing MCE to increase the attractive force. Epidermal MCE could be worn long enough to optimize the external (and/or internal) position before implanting the MCE at a targeted location subcutaneously and/or in the pleural space.

In another method of use, the external MCE could be large permanent or electromagnets, preferably configured in an array, positioned above the patient (not necessarily directly on the skin) which are rotated or otherwise electromagnetically steered to pull internal and/or pleural MCE toward or away from one another. Using the attractive force of the MCE within the lung, the strength and/or direction of the external magnetic array could be steered to manipulate the lung position for optimal lung compression. This could be used to guide the placement of additional internal or external MCE.

To facilitate removal of implanted MCE, it may be necessary to first reduce the magnetic force between the elements. One means of accomplishing this would be to heat a given MCE to near its Curie Temperature. A forceps with positive and negative conductive elements could be attached to the MCE and a direct or alternating current applied across the MCE to resistively heat it. An inductive current could also be applied within the graspers to inductively heat the MCE. The patient could also be exposed to a larger external electric field to inductively heat the MCE. An internal temperature sensor could be placed adjacent the elements to provide feedback on the field strength so as not to create heating that could result in clinical sequela exceeding the risk/benefit to the patient. Another method would be to apply an alternating current directly through the magnet to reorient the dipoles. Similarly, AC current passing through a solenoid in proximity to the magnet could produce a rapidly fluctuating magnetic field to reorient the dipoles. Subjecting the magnets to an MRI field would also demagnetize them.

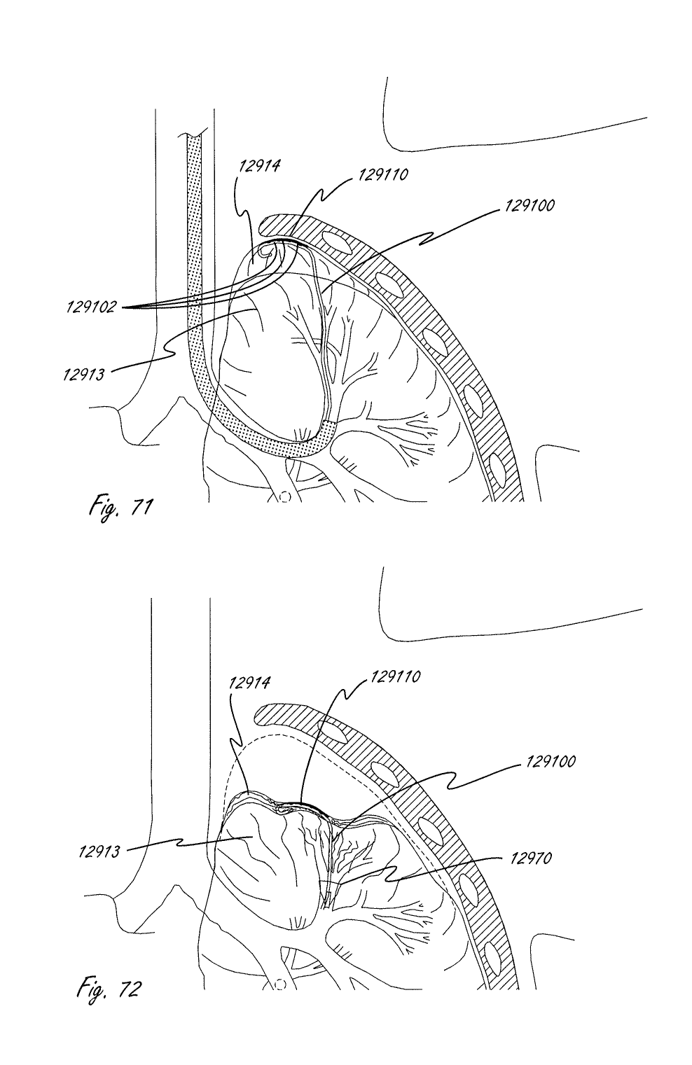

FIGS. 71 and 72 provide an embodiment similar to that described in FIGS. 70-79 of WO 2016/115193 A1, where an atraumatic catheter 129100 may be advanced into a distal diseased lung tissue region 12914. This region 12914 is considered more friable and open than other healthy or diseased regions of lung 12913. The regions 12913, while potentially having alveolar and parenchymal damage, still have intact airways passing through. The region 12914 may also be considered to be a pulmonary bullae or bleb. In this embodiment, the catheter 129100 is sized to pass through a distal airway of about 1-2 mm diameter and also has a floppy atraumatic tip such that it does not puncture the friable tissue. The diameter of the distal end may be tapered smaller for access through smaller airway sizes (0.5-1 mm diameter). The catheter 129100 is advanced by itself or with the aid of a removable floppy guidewire (not shown), which may also have a tapered outer diameter to match the catheter until it slides along the inside of the tissue on the outer border of the lung. A shaped stylet may be additionally or alternatively inserted through a separate lumen of the catheter to direct the catheter to the tissue wall. The catheter 129100 has a plurality of orifices 129102 spaced along a length (4 cm nominal but ranging 1-20 cm) of the distal portion. An orifice may optionally be provided at the distal tip. In some embodiments, the orifices may be formed from a porous material through which the adhesive uniformly “weeps”, such as sintered metal, ePTFE, or small laser drilled holes in the tubing. In others the orifices are more discreet, such as openings of 0.005″ to up to half the diameter of the catheter. The guidewire, if used, may be removed after the catheter 129100 is in position. Any stylet may or may not be removed. A biocompatible adhesive liquid or gel 129110 may be injected through the catheter 129100 from the proximal end such that it exits orifices 129102 at the end of the catheter 129100 and quickly cures against the tissue. Curing may be moisture activated, such as by many cyanoacrylate adhesives known in the art, or UV activated by a fiberoptic light source integrated into the catheter, by a setup of a two part mix, or any combination thereof. The adhesive and catheter preferably contain a radiopaque compound for visibility under fluoroscopy, which also provides a means for the user to control the amount of adhesive delivered. In some embodiments, the adhesive may be preloaded in the catheter tip (with leakage prevented with a tight-fitting retractable sleeve), and pushed out through orifices with compressed air or saline. A proximal anchor may be deployed in a more proximal airway, through which the catheter 129100 may be pulled through. Tensioning of the catheter 129100 pulls the friable tissue 12914 downward against the firmer tissue 12913 and the two together may be further compressed. The catheter 12914 may be secured to the proximal anchor 12970 by any number of mechanical means and/or injection of additional adhesive or an otherwise curable material. The remaining length of the catheter proximal to the proximal anchor may be trimmed or mechanically detached and removed.

Adhesion of the catheter to the tissue may be reversed by using an adhesive material that is deactivated with a light source such as UV or IR, or by administering a biocompatible solvent to the adhesive.

The above catheter adhesion method may also be accomplished with a plurality of individual catheters. The catheters may also be configured into arms of a spline which is mechanically compressed to expand within the tissue region 12914 until an acceptable number of arms are in contact with the tissue for adhesive delivery. The arms are then tensioned to pull down against one another and collapse the tissue region 12914.

1. A method of treating COPD, comprising:
bronchoscopically positioning a lung anchor in lung tissue;
positioning an outer implant member in pleural space proximate the lung anchor; and
coupling the lung anchor to the outer implant member and thereby compressing lung tissue.

2. The method of claim 1, wherein coupling the lung anchor to the outer implant member comprises magnetically coupling the lung anchor to the outer implant member.

3. The method of claim 1, wherein bronchoscopically positioning a lung anchor in lung tissue comprises bronchoscopically positioning a lung anchor with a tether extending proximally from the lung anchor.

4. The method of claim 3, wherein the lung anchor is a first lung anchor, the method further comprising bronchoscopically delivering a second anchor in the lung tissue, proximal to the first lung anchor, where the tether line extends from the proximal anchor.

5. The method of claim 4, further comprising securing the second anchor in the lung tissue, and wherein the coupling step occurs before securing the second anchor in the lung tissue.

6. The method of claim 5, further comprising tensioning the tether line and securing the tether line to the second anchor, the tensioning step occurring after the coupling step.

7. The method of claim 4, further comprising securing the second anchor in the lung tissue, and wherein the coupling step occurs after securing the second anchor in the lung tissue.

8. The method of claim 7, further comprising tensioning the tether line and securing the tether line to the second anchor, the tensioning step occurring before the coupling step

9. The method of claim 1 wherein positioning an outer implant member in pleural space proximate the lung anchor comprises advancing a pleural delivery catheter into the pleural space.

10. The method of claim 9, wherein advancing a pleural delivery catheter into the pleural space comprises introducing the pleural delivery catheter via a thoracostomy.

11. The method of claim 9, wherein the coupling step comprises moving at least a portion of the outer implant member closer to the lung anchor.

12. The method of claim 11, wherein moving at least a portion of the outer implant member comprises deflecting the pleural delivery catheter.

13. The method of claim 1, wherein the coupling step comprises magnetically coupling the lung anchor to the outer implant member from a magnetic attraction between magnetic coupling elements provided in the lung anchor and outer implant member.
